(19) United States
(12) Patent Application Publication
(10) Pub. No.: US 2012/0204291 A1
(43) Pub. Date: Aug. 9, 2012

(54) STRESS TOLERANT PLANTS

(75) Inventors: Nestor Carrillo, Santa Fe (AR); Mariana Giro, Rosario (AR); Anabella Fernanda Lodeyro, Rosario (AR); Matias Daniel Zurbriggen, Freiburg (DE)

(73) Assignee: Plant Bioscience Limited, Norfolk (GB)

(21) Appl. No.: 13/389,665

(22) PCT Filed: Aug. 11, 2010
(86) PCT No.: PCT/GB10/51332

§ 371 Date: Apr. 19, 2012

(30) Foreign Application Priority Data
Aug. 11, 2009 (GB) 0914012.0

(51) Int. Cl. A01H 5/00; A01H 5/10; C12N 5/10; A01H 1/06; C12N 15/82

(52) U.S. Cl. 800/279; 800/278; 800/289; 800/298; 800/317.3; 800/320; 435/320.1; 435/419

(57) ABSTRACT

The invention relates to methods for increasing stress tolerance in plants by expressing a nucleic acid encoding a FId polypeptide and a nucleic acid sequence encoding a FNR polypeptide in a plant.

### FIELD OF THE INVENTION

The invention relates to method for producing plants with increased tolerance to stress, in particular oxidative stress. The invention also relates to gene expression constructs for use in such methods and to transgenic plants with increased tolerance to stress, for example plants obtained or obtainable by the methods described herein.

### INTRODUCTION

External conditions that adversely affect growth, development or productivity trigger a wide range of plant responses, such as altered gene expression, cellular metabolism and changes in growth rates and crop yields. There are two types of stress: biotic stress is imposed by other organisms, such as a pathogen, whereas abiotic stress arises from an excess or deficit in the physical or chemical environment, such as drought, salinity, high or low temperature or UV light.

Environmental stress is a major limiting factor for plant productivity and crop yield. When plant cells are under environmental stress, several chemically distinct reactive oxygen species (ROS) are generated by partial reduction of molecular oxygen and these can cause oxidative damage or act as signals. Auto-oxidation of components of the photosynthetic electron transport chain leads to the formation of superoxide radicals and their derivatives, hydrogen peroxide and hydroxyl radicals. These compounds react with a wide variety of biomolecules including DNA, causing cell stasis and death (Kim et al 2008, Vranová et al 2002).

Flavodoxin (FId) is an electron transfer flavoprotein found in bacteria and some marine algae, but not in plants (Zurbriggen et al., 2007), which is able to efficiently engage in several ferredoxin (Fd)-dependent oxido-reductive pathways, including photosynthesis, nitrogen assimilation and thioredoxin-mediated redox regulation (Tognetti et al., 2006, 2007b). FId levels are up-regulated in microorganisms exposed to oxidative and abiotic stresses (Singh et al., 2004). When expressed in plant chloroplasts, the flavoprotein behaves as a general antioxidant preventing formation of different types of ROS in chloroplasts (Tognetti et al., 2006). The resulting transgenic plants developed multiple tolerance to a wide range of environmental challenges, redox-cycling oxidants and xenobiotics (Tognetti at al., 2006; 2007a, PCT/GB2002/004612 all of which incorporated herein by reference). In iron-starved cyanobacteria, FId is reduced by photosystem I (PSI), as it occurs in the FId transformed plants (Tognetti et al., 2006). In heterotrophic bacteria, FId can be reduced by a pyruvate-FId reductase and by an NADPH-FId reductase (Blaschkowski et al., 1982). FId also accumulates constitutively in cyanobacterial heterocysts and it has been argued that it could participate in electron transfer to nitrogenase (Sandmann et al., 1990), but the nature of the ultimate electron donor is unknown and the induction of a more efficient, heterocyst-specific ferredoxin that could mediate this reaction has cast doubts on the role of FId during dinitrogen fixation (Razquin et al., 1995).

Ferredoxin-NADP(H) reductase (FNR) (EC 1.18.1.2) is a thylakoid bound enzyme in both plants and cyanobacteria, engaged in a physical, constitutive manner in electron transfer from ferredoxin or FId to NADP⁺ for NADPH formation (Carrillo and Ceccarelli, 2003). This activity directly collides with the possibility of mediating the opposite reaction in light, when there is strong electron pressure from PSI. Thus, it is unlikely that FNR-mediated reduction of ferredoxin (or FId) by NADPH occurs in vivo at any significant rate, and no observation on such an activity has been reported so far. However, solubilised FNR becomes uncoupled with the rest of the chain and readily catalyses it (Carrillo and Ceccarelli, 2003). Indeed, soluble FNR is almost inactive in mediating NADP⁺photoreduction by isolated, FNR-depleted thylakoids (Forti and Bracale, 1984). In cyanobacterial species which contain phycobilisomes for light harvesting, FNR is made up of three domains: an N-terminal domain involved in phycobilisome attachment, followed by an FAD-binding domain and an NADP(H)-binding domain which together constitute the active part of the enzyme (Carrillo and Ceccarelli, 2003). An alternative initiation codon is located at the beginning of the second domain to yield a two-domain soluble FNR (Thomas et al., 2006). This internal

Met is used preferably when cells are shifted to a heterotrophic lifestyle and the ability to transfer electrons from NADPH to Fd or FId is required (Thomas et al., 2006). A scheme describing the theoretical model is provided in FIG. 1. The enzyme is found in all cyanobacteria and photosynthetic eukaryotic cells. Other enzymes with a similar specificity but different physiological roles have been described in several non-photosynthetic plant tissues, in mammalian mitochondria and in several bacteria. Cyanobacterial FNR has been well characterized (Sancho, 1987, Schluchter 1992). Moreover, the petH gene coding for FNR has been cloned from several cyanobacterial strains (Fillat et al., 1993). The presence of active FNR can be detected by diaphorase activity assays as described below.

It is therefore known that incorporation of a bacterial FId into tobacco chloroplasts can compensate for the decline in Fd levels, leading to increased tolerance to oxidants and to a wide range of adverse stress conditions. The present invention is aimed at improving stress tolerance in plants by ensuring that FId is maintained in a reduced condition.

### SUMMARY OF THE INVENTION

In one aspect, the invention relates to a method for producing a plant with enhanced stress tolerance comprising expressing a nucleic acid sequence encoding a flavodoxin polypeptide and a nucleic acid sequence encoding a ferredoxin NADP(H) reductase polypeptide in a plant. A plant obtained or obtainable by such method is also within the scope of the invention. The invention further relates to a nucleic acid construct comprising a gene sequence wherein said gene sequence comprises a nucleic acid sequence encoding a cyanobacterial FNR and a chloroplast targeting sequence. In another aspect, the invention relates to a nucleic acid construct comprising a nucleic acid sequence encoding a flavodoxin polypeptide (FId) and a nucleic acid sequence encoding a ferredoxin NADP(H) reductase (FNR) polypeptide. In a further aspect, the invention relates to a transgenic plant with enhanced stress tolerance expressing a nucleic acid sequence encoding a flavodoxin polypeptide and a nucleic acid sequence encoding a ferredoxin NADP(H) reductase polypeptide. In another aspect, the invention relates to a method for reducing the amount of ROS in a plant in response to stress comprising expressing a flavodoxin polypeptide and a ferredoxin NADP(H) reductase polypeptide in a plant.

### FIGURES

The invention is further illustrated in the non-limiting figures.

FIG. 1. Proposed electron route in double transformants expressing FId and FNR from cyanobacteria. Under normal growth conditions (top panel), both ferredoxin (Fd) and FId could mediate electron transfer to productive routes, Fd being probably preferred on efficiency grounds. Under stress (bottom panel), Fd levels decline and FId takes over photosynthetic electron transfer to NADP, while soluble FNR uses part of the NADPH formed to keep FId reduced, preventing ROS formation and closing the virtuous cycle.

FIG. 2. FNR accumulation in leaves of tobacco wild type (PH) and transformants. Leaf extracts from 6-week-old independent transformed plants (pnn) corresponding to 17 μg protein were fractionated by SDS-PAGE and blotted onto nitrocellulose membranes for immunodetection of FNR with antisera directed against the Anabaena reductase.

FIG. 3. Subcellular localisation and in-gel diaphorase activity of FNR from transgenic tobacco plants. A) Thylakoids and stroma were separated after osmotic shock of isolated intact chloroplasts from wild-type and pFNR plants. Samples corresponding to 4 μg chlorophyll were resolved by SDS-PAGE and the presence of FNR was determined by immunoblot analysis. B) Stroma from wild-type and pFNR plants, corresponding to 15 μg of total soluble protein, were resolved by native electrophoresis and stained for diaphorase activity.

FIG. 4. Expression levels of FNR and FId in the progeny of X4 plants. Leaf extracts from 6-week-old tobacco plants corresponding to 8 μg of total soluble protein were fractionated by SDS-PAGE and blotted onto nitrocellulose membranes for immunodetection with antisera directed against the Anabaena FNR and FId.

FIG. 5. Effect of methyl viologen (MV) on leaf discs of FNR/FId expressing plants. Leaf discs from 6-week old tobacco plants were placed on 20 μM MV and illuminated at 600 μmol quanta m⁻² s⁻¹. A) Picture taken after 7 h of incubation. B) Ion leakage was estimated by measuring the increase in relative conductivity of the medium after MV treatment of leaf discs. C) Chlorophylls and carotenoids were determined after 7 h of MV treatment.

FIG. 6. Effect of MV on whole tobacco plants. Four-week old plants were transferred to a hydroponics system. Pictures of leaves were taken after 24 h of exposure to 100 μM MV under growth chamber conditions.

FIG. 7. A) Detection of lipid peroxides. Leaf discs from 6-week-old plants were placed on 10 μM MV or water (right hand bar) and illuminated at 700 μmol quanta m⁻² s⁻¹ during 3 h. Each value is a mean of four sample replicate measurements±standard deviation. B) APX activity of leaf extracts from discs of FNR/FId expressing plants. Leaf discs from 6-week old tobacco plants were placed on 20 μM MV and illuminated at 600 μmol quanta m⁻² s⁻¹. Samples were taken after 1.5 and 3 h of incubation.

FIG. 8. Scheme of the binary vector pCAMBIA 2200 containing a fragment of the in-frame fusion between the sequences encoding pea FNR transit peptide and the flavodoxin gene. The cassette inserted in the Eco RI site of the pCAMBIA 2200 was previously constructed in pDH51. This Eco RI fragment contained the CaMV 35S promoter, the flavodoxin chimeric gene and the CaMV35S polyadenylation signal.

FIG. 9. Scheme of the binary vector pCAMBIA 2200 containing a fragment of the in-frame fusion between the sequences encoding pea FNR transit peptide and the two C-terminal domains of the Anabaena FNR gene. The cassette inserted in the Eco RI site of the pCAMBIA 2200 was previously constructed in pDH51. This Eco RI fragment contained the CaMV 35S promoter, the FNR chimeric gene and the CaMV35S polyadenylation signal.

FIG. 10. Scheme of the Multisite Gateway derived binary vector pBinary-BRACT B1,4-ubi-FNR/B2,3-actin-FId containing the in-frame fusions between the sequence encoding a pea FNR transit peptide and the two C-terminal domains of the FNR (TP-FNR), and the FId (TP-FId) genes from Anabaena PCC7119. The TP-FNR and TP-FId constructs are flanked in the co-expression vector by the nos polyadenylation signal and the ubi and actin promoters, respectively. These constructs are first cloned into appropriate donor vectors of the pDONR221 vector series by site-specific BP recombination reactions. The resulting entry clones are engaged in turn in a simultaneous double LR site-specific recombination with a customized binary T-DNA MultiSite Gateway destination vector, namely pDEST-BRACT R1,4-ubi/R2,3-actin, yielding the expression clone pBinary-BRACT B1,4-ubi-FNR/B2,3-actin-FId which comprises the two genes of interest. The cloning strategy of the constructs into the binary vector is based on the BP and LR site-specific recombination reactions of the Multisite Gateway technology (Invitrogen, http://www.invitrogen.corn). Hyg: Selection marker (resistance to hygromicin); LB: left border; nos: nopaline synthase; RB: right border; TP: transit peptide; ubi: ubiquitin.

FIG. 11. Construction of binary vectors for the co-expression of FId and FNR polypeptides in plants. The schematic figure exhibits the construction of the pBinary-BRACT B1,4-ubi-FNR/B2,3-actin-FId binary vector for the co-expression of FNR and FId in plants. The PCR products of the sequences encoding the chimeric fusions of FNR and FId to a chloroplast targeting transit peptide (TP) flanked by attB site-specific recombination sequences (attB1-FNR-attB4 and attB2-FId-attB3, respectively) are substrates in a BP recombination reaction with the appropriate donor vectors (pDONR21 P1-P4 and pDONR p2-P3, respectively). The resulting pENTR221 L1-L4-FNR and pENTR221 L2-L3-FId entry clones are engaged in turn in a simultaneous double LR site-specific recombination with a customized binary T-DNA MultiSite Gateway destination vector, namely pDEST-BRACT R1,4-ubi/R2,3-actin, giving forth an expression clone comprising the two genes of interest under the control of constitutive promoters. The procedure is performed according to the protocols, instructions and nomenclature suggested by the manufacturer (Invitrogen, http://ww.invitrogen.com). ccdB: gene used for negative selection of the vector; LB: left border; nos: nopaline synthase; RB: right border; TP: transit peptide; ubi: ubiquitin.

FIG. 12. Barley Stress. Effect of methyl viologen (MV) on leaf strips of FNR/FId-expressing heterozygous barley plants. Leaf strips of 10-15 mm length were cut from leaves of 6-week old barley plants grown in soil. Leaf stripes were then incubated in 50 μM MV and 0.05% Tween-20 for 30 minutes at 20° C. in the dark to allow diffusion of the MV into the tissue. The strips were then placed with the adaxial side up in plastic trays a 450 μmol quanta m⁻² s⁻¹ light source. Controls were kept in distilled water containing 0.05% Tween-20. A) Chlorophyll and B) carotenoid contents were estimated after 7.5 h of illumination. FNR (1×): transgenic barley heterozygous for FNR. FId (1×): transgenic barley heterozygous for FId. FNR/FId (1×): transgenic barley heterozygous for FNR and FId. WT: wild-type barley.

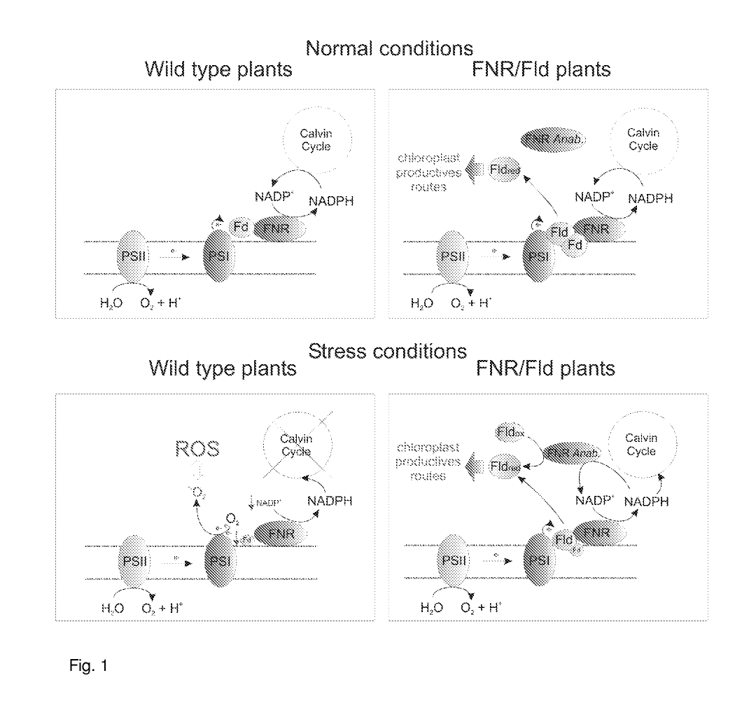

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature.

As mentioned above, it is known that incorporation of a bacterial flavodoxin (FId) into tobacco chloroplasts can compensate for the decline in Fd levels, leading to increased tolerance to oxidants and to a wide range of adverse stress conditions. The present inventors have surprisingly found that introducing a second gene derived from bacteria having a FId-reducing activity into a plant expressing bacterial FId can improve the stress tolerance of the plant. Without wishing to be bound by theory, the inventors believe that this is due to maintaining FId in a reduced condition. As shown in the examples, the inventors have used a construct with a nucleic acid sequence derived from a cyanobacterium and encoding a chloroplast-targeted ferredoxin NADP(H) reductase (FNR) polypeptide and expressed said bacterial gene in a plant expressing chloroplast-targeted FId.

Thus, in one aspect, the invention relates to a method for producing a plant with enhanced stress tolerance comprising expressing a nucleic acid sequence encoding a FId polypeptide and a nucleic acid sequence encoding a FNR polypeptide in a plant. Expression of these sequences in a plant according to the invention can be achieved in different ways as explained herein.

In a first embodiment, the method comprises expressing a nucleic acid construct that directs the co-expression of FId polypeptide and FNR as described herein in a plant. Thus, a single construct according to the different embodiments as detailed herein can direct the co-expression of both genes in a plant transformed with such construct according to the different aspects of the invention. The resulting transgenic plant produces FId and FNR polypeptides. In this method, a plant is transformed with the co-expression construct and stable homozygous plants expressing both transgenes are generated and selected.

The construct that can be used in this method is described in detail below.

The nucleic acid construct comprises a nucleic acid sequence encoding a FId polypeptide and a nucleic acid sequence encoding a FNR polypeptide. Preferably, the FId and FNR sequences are of bacterial origin.

In one embodiment, the nucleic acid sequence encoding a FId polypeptide is derived from a cyanobacterium and the flavodoxin polypeptide is a cyanobacterial flavodoxin. Alternatively, the nucleic acid sequence encoding a FId polypeptide is derived from a heterotrophic bacterium. The cyanobacterium may be selected from Crocosphaera, Cyanobium, Cyanothece, Microcystis, Synechococcus, Synechocystis, Thermosynechococcus, Microchaetaceae, Nostocaceae, Lyngbya, Spirulina or Trichodesmium. Preferred genera include Synechococcus, Fremyella, Tolypothrix Anabaena, Anabaenopsis, Aphanizomenon, Aulosira, Cylindrospermopsis, Cylindrospermum, Loefgrenia, Nodularia, Nostoc or Wollea. Preferably, the genus is Anabaena and the cyanobacterium is Anabaena PCC7119 (Fillat et at 1990).

In one embodiment, the FId sequence has a nucleic acid sequence selected from the sequences as shown in table 1 below. In one embodiment, the FNR sequence has a nucleic acid sequence elected from the sequences as shown in table 2 below.

TABLE 1

| Accession No | | Gene name | Organism |
|---|---|---|---|
| NP_358768.1 | gi\|15903218 | Flavodoxin | Streptococcus |
| | | | pneumoniae R6 |
| NP_345761.1 | gi\|15901157 | Flavodoxin | Streptococcus |
| | | | pneumoniae TIGR4 |
| NP_311794.1 | gi\|15833021 | flavodoxin 2 | Escherichia coli |
| | | | 0157:_H7] |
| NP_311593.1 | gi\|15832820 | putative flavodoxin | Escherichia coli |
| | | | 0157:_H7 |
| NP_308742.1 | gi\|15829969 | flavodoxin 1 | Escherichia coli |
| | | | O157:_H |
| CAC92877.1 | gi\|15980620 | flavodoxin 1 | Yersinia pestis |
| CAC89737.1 | gi\|15978964 | flavodoxin 2 | Yersinia pestis |
| NP_350007.1 | gi\|15896658 | Flavodoxin | Clostridium |
| | | | acetobutylicum |
| NP_349066.1 | gi\|15895717 | Flavodoxin | Clostridium |
| | | | acetobutylicum |
| NP_347225.1 | gi\|15893876 | Flavodoxin | Clostridium |
| | | | acetobutylicum |
| NP_346845.1 | gi\|15893496 | Flavodoxin | Clostridium |
| | | | acetobutylicum |
| NP_348645.1 | gi\|15895296 | Predicted | Clostridium |
| | | flavodoxin | acetobutylicum |
| NP_347225.1 | gi\|15893876 | Flavodoxin | Clostridium |
| | | | acetobutylicum |
| NP_346845.1 | gi\|15893496 | Flavodoxin | Clostridium |
| | | | acetobutylicum |
| NP_282528.1 | gi\|15792705 | Flavodoxin | Campylobacter |
| | | | jejuni |
| AAK28628.1 | gi\|13507531 | Flavodoxin | Aeromonas |
| | | | hydrophila |
| NP_268951.1 | gi\|15674777 | putative flavodoxin | Streptococcus |
| | | | pyogenes |
| NP_266764.2 | gi\|15672590 | Flavodoxin | Lactococcus lactis |
| | | | subsp. lactis |
| NP_207952.1 | gi\|15645775 | flavodoxin (fldA) | Helicobacter pylori |
| | | | 26695 |
| NP_232050.2 | gi\|15642417 | flavodoxin 2 | Vibrio cholerae |
| NP_231731.1 | gi\|15642099 | flavodoxin 1 | Vibrio cholerae |
| NP_219360.1 | gi\|15639910 | Flavodoxin | Treponema |
| | | | pallidum |
| NP_240122.1 | gi\|15616909 | Flavodoxin | 1 Buchnera sp. APS |
| NP_214435.1 | gi\|15607053 | Flavodoxin | Aquifex aeolicus |
| FXAVEP | gi\|625194 | Flavodoxin | Azotobacter |
| | | | vinelandii |
| S38632 | gi\|481443 | flavodoxin | -Synechocystis sp. |
| | | | (strain PCC 6803) |
| FXDV | gi\|476442 | flavodoxin | Desulfovibrio |
| | | | vulgaris |
| A34640 | gi\|97369 | flavodoxin | Desulfovibrio |
| | | | salexigens |
| S24311 | gi\|97368 | flavodoxin | Desulfovibrio gigas |
| | | | (ATCC 19364) |
| A37319 | gi\|95841 | flavodoxin | A Escherichia coli |
| S06648 | gi\|81145 | flavodoxin | red alga (Chondrus |
| | | | crispus) |
| S04600 | gi\|79771 | flavodoxin | Anabaena variabilis |
| A28670 | gi\|79632 | flavodoxin | Synechococcus sp |
| S02511 | gi\|78953 | flavodoxin | Klebsiella |
| | | | pneumoniae |
| FXDVD | gi\|65884 | flavodoxin | Desulfovibrio |
| | | | desulfuricans |
| | | | (ATCC 29577) |
| FXCLEX | gi\|65882 | flavodoxin | Clostridium sp |
| FXME | gi\|6588I | flavodoxin | Megasphaera |
| | | | elsdenii |
| NP_071157.1 | gi\|11499913 | flavodoxin, | Archaeoglobus |
| | | putative | fulgidus |
| BAA17947.1 | gi\|1653030 | flavodoxin | Synechocystis sp. |
| | | | PCC 6803 |
| BAB61723.1 | gi\|14587807 | Flavodoxin 2 | Vibrio fischeri |
| BAB61721.1 | gi\|14587804 | Flavodoxin 1 | Vibrio fischeri |
| AAK66769.1 | gi\|14538018 | flavodoxin | Histophilus ovis |
| P57385.1 | gi\|11132294 | FLAVODOXIN | |
| AAC75933.1 | gi\|1789262 | flavodoxin 2 | Escherichia coli |
| | | | K12 |
| AAC73778.1 | gi\|1786900 | flavodoxin 1 | Escherichia coli |
| | | | K12 |
| AAC75752.1 | gi\|1789064 | putative flavodoxin | Escherichia coli |
| | | | K12 |
| F69821 | gi\|7429905 | flavodoxin | Bacillus subtilis |
| | | homolog yhcB | |
| QQKBFP | gi\|2144338 | pyruvate | Klebsiella |
| | | (flavodoxin) | pneumoniae |
| | | dehydrogenase | |
| | | nifJ | |
| S16929 | gi\|95027 | flavodoxin A | Azotobacter |
| | | | chroococcum |
| F71263 | gi\|7430914 | probable | Syphilis spirochete |
| | | flavodoxin | |
| A64665 | gi\|7430911 | flavodoxin | Helicobacter |
| | | | pylori_(strain |
| | | | 26695 |
| JE0109 | gi\|7430907 | | Desulfovibrio |
| | flavodoxin | | vulgaris |
| S42570 | gi\|628879 | flavodoxin | Desulfovibrio |
| | | | desulfuricans |
| | | | (ATCC |
| BAB13365.1 | gi\|10047146 | flavodoxin | Alteromonas sp. 0-7 |
| AAF34250.1 | gi\|6978032 | flavodoxin | Desulfovibrio gigas |
| CAB73809.1 | gi\|6968816 | flavodoxin | Campylobacter |
| | | | jejuni |
| D69541 | gi\|7483302 | flavodoxin | homolog |
| | | | Archaeoglobus |
| | | | fulgidus |
| F70479 | gi\|7445354 | flavodoxin | Aquifex aeolicus |
| S55234 | gi\|1084290 | flavodoxin isoform I | Chlorella fusca |
| S18374 | gi\|2117434 | flavodoxin | Anabaena sp. (PCC |
| | | | 7119) |
| 555235 | gi\|1084291 | flavodoxin isoform | Chlorella fusca |
| | | II | |
| C64053 | gi\|1074088 | flavodoxin A | Haemophilus |
| | | | influenzae (strain |
| | | | Rd KW20) |
| A61338 | gi\|625362 | flavodoxin | Clostridium |
| | | | pasteurianum |
| A39414 | gi\|95560 | flavodoxin | Enterobacter |
| | | | agglomerans |
| | | | plasmid |
| AAD08207.1 | gi\|2314319 | flavodoxin (fldA) | Helicobacter pylori |
| | | | 26695 |
| CAB37851.1 | gi\|4467982 | flavodoxin | Rhodobacter |
| | | | capsulatus |
| AAC65882.1 | gi\|3323245 | flavodoxin | Treponema |
| | | | pallidum |
| AAB88920.1 | gi\|2648181 | flavodoxin, | Archaeoglobus |
| | | putative | fulgidus |
| AAB65080.1 | gi\|2289914 | flavodoxin | Klebsiella |
| | | | pneumoniae |
| AAB53659.1 | gi\|710356 | flavoprotein | Methano- |
| | | | thermobacter |
| | | | Thermautotrophicus |
| AAB51076.1 | gi\|1914879 | flavodoxin | Klebsiella |
| | | | pneumoniae |
| AAB36613.1 | gi\|398014 | flavodoxin | Azotobacter |
| | | | chroococcum |
| AAB20462.1 | gi\|239748 | flavodoxin | Anabaena |
| AAA64735.1 | gi\|142370 | flavodoxin_(nifF) | Azotobacter |
| | | | vinelandii |
| BAA35341.1 | gi\|1651296 | Flavodoxin | Escherichia coli |
| BAA35333.1 | gi\|1651291 | Flavodoxin | Escherichia coli |
| AAA27288.1 | gi\|415254 | flavodoxin | Synechocystis sp. |
| AAA27318.1 | gi\|154528 | Flavodoxin | Synechococcus sp. |
| AAC45773.1 | gi\|1916334 | putative flavodoxin | Salmonella |
| | | | typhimurium |
| AAC07825.1 | gi\|2984302 | flavodoxin | Aquifex aeolicus |
| AAC02683.1 | gi\|2865512 | flavodoxin | Trichodesmium |
| | | | erythraeum |

TABLE 2

| Accession No | | Gene name | Organism |
|---|---|---|---|
| P21890.2 | gi/585127 | petH | Anabaena sp. (strain PCC |
| | | | 7119) |
| P58558.1 | gi/20138171 | petH (all4121) | Anabaena sp. (strain PCC |
| | | | 7120) |
| Q44549.1 | gi/2498066 | petH (Ava_0782) | Anabaena variabilis (strain |
| | | | ATCC 29413/PCC 7937) |
| P00454.1 | gi/119907 | petH | Spirulina sp. |
| P31973.1 | gi/399488 | petH | Synechococcus sp. (strain |
| | | (SYNPCC7002_A0853) | ATCC 27264/PCC 7002/PR- |
| | | | 6) (Agmenellum |
| | | | quadruplicatum) |
| Q55318.2 | gi/2498067 | petH (slr1643) | Synechocystis sp. (strain PCC |
| | | | 6803) |
| Q93RE3.1 | gi/29839385 | petH (tlr1211) | Thermosynechococcus |
| | | | elongatus (strain BP-1) |
| ZP_01619151.1 | gi/119484669 | L8106_14390 | Lyngbya sp. PCC 8106 |
| ZP_01629813.1 | gi/119510685 | N9414_21973 | Nodularia spumigena CCY |
| | | | 9414 |
| ZP_01730168.1 | gi/126659027 | CY0110_28804 | Cyanothece sp. CCY 0110 |
| ZP_01086181.1 | gi/87303393 | WH5701_10210 | Synechococcus sp. WH 5701 |
| ZP_01080624.1 | gi/87124776 | RS9917_01102 | Synechococcus sp. RS9917 |
| ZP_01124447.1 | gi/88808938 | WH7805_04581 | Synechococcus sp. (strain |
| | | | WH7805) |
| YP_00122583.1 | gi/148239896 | petH | Synechococcus sp. (strain |
| | | (SynWH7803_1560) | WH7803) |
| YP_001227016.1 | gi/148241859 | petH | Synechococcus sp. (strain |
| | | (SynRCC307_0760) | RCC307) |
| CAO86244.1 | gi/15902595 | IPF_5476 | Microcystis aeruginosa PCC |
| | | | 7806 |
| YP_001656271.1 | gi/166363998 | petH (MAE_12570) | Microcystis aeruginosa (strain |
| | | | NIES-843) |
| YP_001802411.1 | gi/172035910 | petH (cce_0994) | Cyanothece sp. (strain ATCC |
| | | | 51142) |
| YP_001866231.1 | gi/186683035 | Npun_R2751 | Nostoc punctiforme (strain |
| | | | ATCC 29133/PCC 73102) |
| BAG48514.1 | gi/190350810 | petH | Nostoc cf. verrucosum |
| BAG48518.1 | gi/190350817 | petH | Nostoc flagelliforme MAC |
| BAG48526.1 | gi/190350832 | petH | Nostoc cf. commune KG-102 |
| ZP_03155450.1 | gi/196256913 | Cyan7822DRAFT_2608 | Cyanothece sp. PCC 7822 |
| ZP_03143292.1 | gi/196244566 | Cyan8802DRAFT_1689 | Cyanothece sp. PCC 8802 |
| YP_002714666.1 | gi/225144671 | S7335_1472 | Synechococcus sp. PCC 7335 |
| BAG69177.1 | gi/197267616 | petH | Nostoc commune IAM M-13 |
| BAG69178.1 | gi/197267618 | petH | Nostoc sp. KU001 |
| BAG69179.1 | gi/197267620 | petH | Nostoc cf. commune SO-42 |
| BAG69180.1 | gi/197267622 | petH | Nostoc carneum IAM M-35 |
| BAG69181.1 | gi/197267624 | petH | Nostoc linckia var. arvense |
| | | | IAM M-30 |
| BAG69182.1 | gi/197267626 | petH | Nostoc sp. (strain PCC 7906) |
| BAG70314.1 | gi/197724770 | petH | Nostoc commune |
| BAG70315.1 | gi/197724772 | petH | Nostoc commune |
| BAG70316.1 | gi/197724774 | petH | Nostoc commune |
| BAG70322.1 | gi/197724786 | petH | Nostoc commune |
| BAG70319.1 | gi/197724780 | petH | Nostoc commune |
| BAG70320.1 | gi/197724782 | petH | Nostoc commune |
| BAG70321.1 | gi/197724784 | petH | Nostoc commune |
| BAG70323.1 | gi/197724788 | petH | Nostoc commune |
| YP_002597543.1 | gi/223491251 | CPCC7001_1059 | Cyanobium sp. PCC 7001 |
| ACJ05621.2 | gi/227438935 | petH | Fremyella diplosiphon B590 |
| ACJ05622.1 | gi/210061096 | petH | Tolypothrix sp. PCC 7601 |
| YP_002372707.1 | gi/218247336 | PCC8801_2543 | Cyanothece sp. (strain PCC |
| | | | 8801) (Synechococcus sp. |
| | | | (strain PCC 8801/RF-1)) |
| YP_002380418.1 | gi/218442089 | PCC7424_5201 | Cyanothece sp. (strain PCC |
| | | | 7424) (Synechococcus sp. |
| | | | (strain ATCC 29155)) |
| ACL47344.1 | gi/21986005 | Cyan7425_5047 | Cyanothece sp. (strain PCC |
| | | | 7425/ATCC 29141) |
| ZP_01470332.1 | gi/116073070 | RS9916_31507 | Synechococcus sp. RS9916 |
| YP_723193.1 | gi/113477132 | Tery_3658 | Trichodesmium erythraeum |
| | | | (strain IMS101) |
| BAE71336.1 | gi/84468507 | petH | Spirulina platensis |
| YP_399995.1 | gi/81299787 | Synpcc7942_0978 | Synechococcus elongatus |
| | | | (strain PCC 7942) (Anacystis |
| | | | nidulans R2) |
| YP_376761.1 | gi/78184326 | Syncc9902_0749 | Synechococcus sp. (strain |
| | | | CC9902) |
| ZP_00516246.1 | gi/67922744 | CwatDRAFT_3658 | Crocosphaera watsonii |
| BAD97809.1 | gi/63002589 | petH | Nostoc commune |
| YP_171276.1 | gi/56750575 | petH (syc0566_c) | Synechococcus sp. (strain |
| | | | ATCC 27144/PCC 6301/ |
| | | | SAUG 1402/1) (Anacystis |
| | | | nidulans) |
| NP-896844.1 | gi/33865285 | petH (SYNW0751) | Synechococcus sp. (strain |
| | | | WH8102) |
| YP_001015330.1 | gi/124026214 | petH (NATL1_15081) | Prochlorococcus marinus |
| | | | (strain NATL1A) |
| YP_291869.1 | gi/72382514 | PMN2A_0675 | Prochlorococcus marinus |
| | | | (strain NATL2A) |
| YP_001009572.1 | gi/123968714 | petH (A9601_11811) | Prochlorococcus marinus |
| | | | (strain AS9601) |
| NP_894932.1 | gi/33863372 | petH (PMT_1101) | Prochlorococcus marinus |
| | | | (strain MIT 9313) |
| YP_001011479.1 | gi/123966398 | petH (P9515_11651) | Prochlorococcus marinus |
| | | | (strain MIT 9515) |
| YP_397581.1 | gi/78779469 | PMT9312_1086 | Prochlorococcus marinus |
| | | | (strain MIT 9312) |
| YP_001016957.1 | gi/124022650 | petH (P9303_09411) | Prochlorococcus marinus |
| | | | (strain MIT 9303) |
| YP_001550998.1 | gi/159903654 | petH (P9211_11131) | Prochlorococcus marinus |
| | | | (strain MIT 9211) |
| YP_001091406.1 | gi/126696520 | petH (P9301_11821) | Prochlorococcus marinus |
| | | | (strain MIT 9301) |
| YP_002672070.1 | gi/225078505 | P9202_860 | Prochlorococcus marinus str. |
| | | | MIT 9202 |
| NP_893192.1 | gi/33861631 | petH (PMM1075) | Prochlorococcus marinus |
| | | | subsp. pastoris (strain |
| | | | CCMP1986/MED4) |
| NP_875515.1 | gi/33240573 | petH (Pro_1123) | Prochlorococcus marinus |
| YP_001516374.1 | gi/158335202 | petH (AM1_2045) | Acaryochloris marina (strain |
| | | | MBIC 11017) |
| BAG48525.1 | gi/190350830 | petH | Nostoc cf. commune KG-54 |
| ZP_01468296.1 | gi/116071027 | BL107_15315 | Synechococcus sp. BL107 |
| YP_730216.1 | gi/113955010 | sync_1003 | Synechococcus sp. (strain |
| | | | CC9311) |
| ABD03802.1 | gi/86558845 | petH (CYB_2882) | Synechococcus sp. (strain JA- |
| | | | 2-3B′a(2-13)) (Cyanobacteria |
| | | | bacterium Yellowstone B- |
| | | | Prime) |
| YP_382213.1 | gi/78213434 | Syncc9605_1917 | Synechococcus sp. (strain |
| | | | CC9605) |
| YP_474703.1 | gi/86605940 | petH (CYA_1257) | Synechococcus sp. (strain JA- |
| | | | 3-3Ab) (Cyanobacteria |
| | | | bacterium Yellowstone A- |
| | | | Prime) |
| ZP_00516246.1 | gi/67922744 | CwatDRAFT_3658 | Crocosphaera watsonii |
| NP_925241.1 | gi/37521864 | petH (gll2295) | Gloeobacter violaceus |

In another embodiment, the nucleic acid sequence encoding a cyanobacterial FId comprises SEQ ID NO. 1. The corresponding amino acid sequence is shown in SEQ ID NO. 6. Variants of SEQ ID NO. 1 or SEQ ID No. 6 are also within the scope of the invention. Variants retain the biological activity of the protein.

In a further aspect, the invention relates to a method for producing a plant with enhanced stress tolerance and methods of increasing stress tolerance of plants comprising expressing a nucleic acid sequence encoding a FNR polypeptide in a plant. Expression of these sequences in a plant according to the invention can be achieved in different ways as explained herein. In another embodiment the FNR polypeptide is polypeptide as represented by SEQ ID NO: 8 or 9, or one shown in table 2 or a cyanobacterial homologue thereof. As shown in the examples, the inventors have used a construct with a nucleic acid sequence derived from a cyanobacterium and encoding a chloroplast-targeted ferredoxin NADP(H) reductase (FNR) polypeptide and expressed said bacterial gene in a plant.

In one embodiment, the nucleic acid sequence encoding a FNR polypeptide is derived from a cyanobacterium and the FNR polypeptide is a cyanobacterial FNR. The cyanobacterium may be a phycobillisome-containing bacterium, for example selected from Crocosphaera, Cyanobium, Cyanothece, Microcystis, Synechococcus, Synechocystis, Thermosynechococcus, Microchaetaceae, Nostocaceae, Lyngbya, Spirulina or Trichodesmium. Preferred genera include Synechococcus, Fremyella, Tolypothrix, Anabaena, Anabaenopsis, Aphanizomenon, Aulosira, Cylindrospermopsis, Cylindrospermum, Loefgrenia, Nodularia, Nostoc or Wollea. In one embodiment, the genus is Anabaena and the cyanobacterium is Anabaena PCC7119 (Fillat et at 1990). Preferably, the sequence comprises a sequence encoding the C-terminal two domain region, but does not comprise the region encoding the phycobillisome- binding domain. For example, the nucleic acid sequence encoding a cyanobacterial FNR comprises SEQ ID NO. 3. The corresponding amino acid sequence is shown in SEQ ID NO. 9. Variants of SEQ ID NO. 3 or SEQ ID No. 9 are also within the scope of the invention. Variants retain the biological activity of the protein.

The construct may be a heterologous gene construct wherein the FId and FNR encoding nucleic acids are derived from different organisms. In another embodiment, both, the FId and FNR encoding nucleic acids are derived from the same organism, for example a cyanobacterium. In one embodiment, both nucleic acid sequences are derived from Anabaena. For example, the construct may comprise the sequences as shown in SEQ ID 1 and 3 or a functional variant thereof.

In a preferred embodiment, the construct described above further comprises at least two chloroplast targeting sequences (encoding a transit peptide) to target each of the polypeptides to the chloroplasts. Any sequence that directs the peptide to the chloroplast is suitable according to the invention. Examples are shown in table 2 of PCT/GB2002/004612 which is incorporated herein by reference. For example, the target sequence may be derived from pea FNR.

Thus, in a preferred embodiment of the invention, the construct may comprise one, preferably both of the sequences as shown in SEQ ID 2 and 4 or a functional variant thereof.

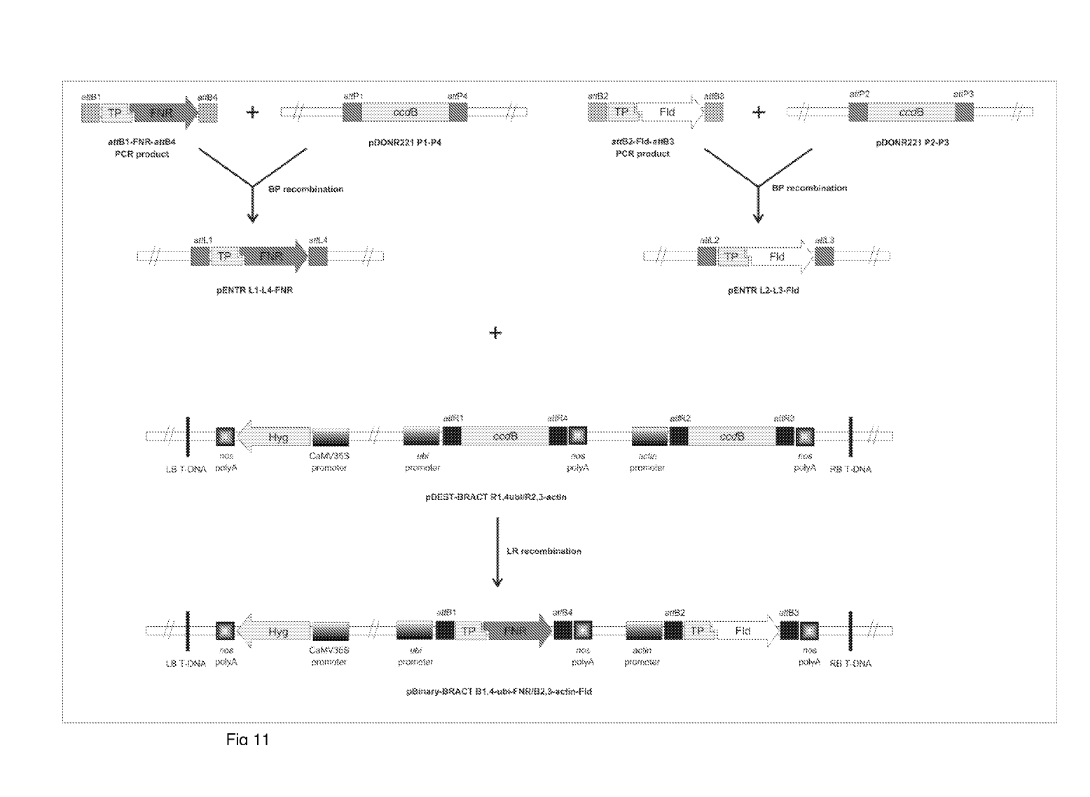

The construct as described above directs the co-expression of nucleic acid sequences encoding the FId and FNR polypeptides from a single construct. Preferably, the construct comprises at least two chloroplast targeting sequences to encode chloroplast targeted polypeptides. As an example, FIG. 10 shows a fusion construct according to the invention and FIG. 11 illustrates how the construct can be made (see also examples).

Constructs as described above are also within the scope of the invention. In other words, the invention relates to a nucleic acid construct comprising both, a nucleic acid sequence encoding a FId polypeptide and a nucleic acid sequence encoding a FNR polypeptide. Various embodiments of the construct and preferred sequences are set out above.

In any of the constructs described herein, wild type sequences that encode FId or FNR polypeptides are preferred, but a mutant/variant sequence or fragments may also be used, provided such sequences encode a polypeptide that has the same biological activity as the wild type sequence. Sequence variations in the wild type sequence include silent base changes that do not lead to a change in the encoded amino acid sequence and/or base changes that affect the amino acid sequence, but do not affect the biological activity of the polypeptide. Changes may be conservative amino acid substitutions, i.e. a substitution of one amino acid residue where the two residues are similar in properties. Thus, variant/mutant polypeptides encoded by such sequences retain the biological activity of the wild type polypeptide and confer stress tolerance. For example, sequence variations in the FNR nucleotide sequence at the following positions (as shown in SEQ ID No. 3) do not appear to affect the activity of the polypeptide: 535: NG; Asn (AAC)/Asp (GAC), 703: A /G; Met (ATG)Nal (GTG), 763: C/G; Gln (CAA)/Glu (GAA). Thus, variants of the FNR nucleic acid sequence/amino acid sequence comprising these alternative nucleotides/amino acids are within the scope of the embodiments of the invention.

Nucleic acids used according to the invention may be double or single stranded, cDNA, genomic DNA or RNA. Any sequences described herein, such as the sequences for the FNR and FId genes can be sequences isolated from a plant, a bacterium or synthetically made sequences. The nucleic acid may be wholly or partially synthetic, depending on design. The skilled person will understand that where the nucleic acid according to the invention includes RNA, reference to the sequence shown should be construed as reference to the RNA equivalent, with U substituted for T.

Additionally, the present invention relates to homologues of the FNR or FLD polypeptide and its use in the method, constructs and vectors of the present invention. The homologue of a FNR or FLD polypeptide has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 8 to 10 or SEQ ID NO: 6 or 7, respectively, and/or represented by its orthologues and paralogues shown in table 2 and table 1, respectively. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides).

According to a further embodiment of the present invention, there are provided methods employing, and constructs, host cells, plants, and vectors comprising,

a) an isolated nucleic acid molecule selected from:
(i) a nucleic acid represented by SEQ ID NO: 1 or 2 or those encoding the homologues listed in table 1;
(ii) the complement of a nucleic acid represented by SEQ ID NO: 1 or 2 or those encoding the homologues listed in table 1;
(iii) a nucleic acid encoding the polypeptide as represented by any one of SEQ ID NO: 6 or 7 or those listed in table 1 preferably as a result of the degeneracy of the genetic code, said isolated nucleic acid can be derived from a polypeptide sequence as represented by (any one of) SEQ ID NO: 6 or 7 or those listed in table 1 and further preferably confers enhanced stress tolerance relative to control plants; (iv) a nucleic acid having, in increasing order of preference at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with any of the nucleic acid sequences of SEQ ID NO: 1 or 2 or those encoding the homologues listed in table 1, preferably to those of SEQ ID NO: 1 or 2, and further preferably conferring enhanced stress tolerance relative to control plants;
(v) a nucleic acid molecule which hybridizes with a nucleic acid molecule of (i) to (iv) under stringent hybridization conditions and preferably confers enhanced stress tolerance relative to control plants;
(vi) a nucleic acid encoding a FLD polypeptide having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by (any one of) SEQ ID NO: 6 or 7 and any of the other amino acid sequences in Table 1 and preferably conferring increased stress tolerance, relative to control plants;
   and
b) an isolated nucleic acid molecule selected from:
(i) a nucleic acid represented by SEQ ID NO: 3 or 4 or those encoding the homologues listed in table 2;
(ii) the complement of a nucleic acid represented by SEQ ID NO: 3 or 4 or those encoding the homologues listed in table 2;
(iii) a nucleic acid encoding the polypeptide as represented by any one of SEQ ID NO: 8 or 9 or those listed in table 2 preferably as a result of the degeneracy of the genetic code, said isolated nucleic acid can be derived from a polypeptide sequence as represented by (any one of) SEQ ID NO: 8 or 9 or those listed in table 2 and further preferably conferring enhanced stress tolerance relative to control plants;
(iv) a nucleic acid having, in increasing order of preference at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with any of the nucleic acid sequences of SEQ ID NO: 3 or 4 or those encoding the homologues listed in table 2, preferably to those of SEQ ID NO: 3 or 4, and further preferably conferring enhanced stress tolerance relative to control plants;
(v) a nucleic acid molecule which hybridizes with a nucleic acid molecule of (i) to (iv) under stringent hybridization conditions and preferably conferring enhanced stress tolerance relative to control plants;
(vi) a nucleic acid encoding a FLD polypeptide having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by (any one of) SEQ ID NO: 8 or 9 and any of the other amino acid sequences in Table 2 and preferably conferring in association with a FLD polypeptide as described herein present in the plants, enhanced stress tolerance relative to control plants.

In a further embodiment there are provided methods employing, and constructs, host cells, plants, and vectors comprising, an isolated nucleic acid molecule selected from
(i) a nucleic acid represented by SEQ ID NO: 3 or 4 or those encoding the homologues listed in table 2;
(ii) the complement of a nucleic acid represented by SEQ ID NO: 3 or 4 or those encoding the homologues listed in table 2;
(iii) a nucleic acid encoding the polypeptide as represented by any one of SEQ ID NO: 8 or 9 or those listed in table 2 preferably as a result of the degeneracy of the genetic code, said isolated nucleic acid can be derived from a polypeptide sequence as represented by (any one of) SEQ ID NO: 8 or 9 or those listed in table 2 and further preferably conferring enhanced stress tolerance relative to control plants;
(iv) a nucleic acid having, in increasing order of preference at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with any of the nucleic acid sequences of SEQ ID NO: 3 or 4 or those encoding the homologues listed in table 2, preferably to those of SEQ ID NO: 3 or 4, and further preferably conferring enhanced stress tolerance relative to control plants;
(v) a nucleic acid molecule which hybridizes with a nucleic acid molecule of (i) to (iv) under stringent hybridization conditions and preferably conferring enhanced stress tolerance relative to control plants;
(vi) a nucleic acid encoding a FLD polypeptide having, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence represented by (any one of) SEQ ID NO: 8 or 9 and any of the other amino acid sequences in Table 2 and preferably conferring in association with a FLD polypeptide as described herein present in the plants, enhanced stress tolerance relative to control plants.

Preferably any comparison to determine sequence identity is performed for polypeptide sequences over the entire polypeptide sequence of any one of SEQ ID NO: 6 to 9, or for nucleic acid sequences over the entire coding region of the nucleic acid sequences of any one of SEQ I D NO: 1 to 4. For example, to determine the sequence identity of a polypeptide sequence to the polypeptide sequence of SEQ ID NO: 8, the sequences are aligned over the entire length of SEQ ID NO: 8.

Control plants are plants not comprising the recombinant FLD and FNR of the invention but in all other ways as identical as possible and treated in the same way as the plants of the invention.

In one embodiment a functional variant of the FLD or FNR polypeptide is a polypeptide with substantially the same biological activity as the FLD as represented by the sequence of SEQ ID NO:6 or 7, or the FNR as represented by the sequence of SEQ ID NO: 8 or 9, respectively. In another embodiment functional variants are polypeptide homologues as defined herein or those encoded by the nucleic acid sequence homologues as defined hereabove.

All nucleic acid constructs as described herein may further comprise a regulatory sequence. Thus, the nucleic acid sequence(s) described herein may be under operative control of a regulatory sequence which can control gene expression in plants. A regulatory sequence can be a promoter sequence which drives the expression of the gene or genes in the construct. For example, the nucleic acid sequence may be expressed using a promoter that drives overexpression. Overexpression according to the invention means that the transgene is expressed at a level that is higher than expression of endogenous counterparts (plant FNR or Fd) driven by their endogenous promoters. For example, overexpression may be carried out using a strong promoter, such as the cauliflower mosaic virus promoter (CaMV35S), the rice actin promoter or the maize ubiquitin promoter or any promoter that gives enhanced expression. Alternatively, enhanced or increased expression can be achieved by using transcription or translation enhancers or activators and may incorporate enhancers into the gene to further increase expression. Furthermore, an inducible expression system may be used, where expression is driven by a promoter induced by environmental stress conditions (for example the pepper pathogen-induced membrane protein gene CaPIMPI or promoters that comprise the dehydration-responsive element (DRE), the promoter of the sunflower HD-Zip protein gene Hahb4, which is inducible by water stress, high salt concentrations and ABA (Dezar et al., 2005), or a chemically inducible promoter (such as steroid- or ethanol-inducible promoter system). Such promoters are described in the art, for example in Pastori (2002). Other suitable promoters and inducible systems are also known to the skilled person.

As a skilled person will know, the construct may also comprise a selectable marker which facilitates the selection of transformants, such as a marker that confers resistance to antibiotics, such as kanamycin.

As detailed above, in one embodiment of the methods of the invention, a single construct is used directing the co-expression of FId and FNr encoding nucleic acid sequences.

In another embodiment, the method for producing a plant with enhanced stress tolerance comprises
a) expressing a nucleic acid construct in a plant said construct comprising a sequence encoding a FId polypeptide,
b) expressing a nucleic acid construct comprising a sequence encoding a FNR polypeptide as described herein,
c) crossing the first and second plant and
d) generating a plant homozygous for and expressing both FNR and FId.

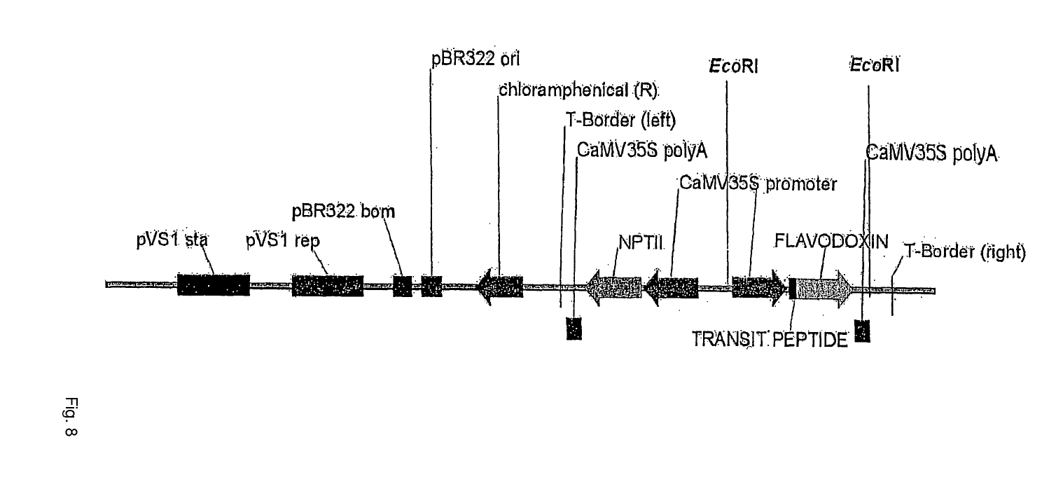

According to the first step of the method, a first plant is transformed with a nucleic acid construct comprising a sequence encoding a flavodoxin polypeptide. Such constructs have been described in Tognetti et al. (2006) and PCT/GB2002/004612, both incorporated herein by reference. Preferred constructs include sequences derived from a cyanobacterium, preferably Anabaena, most preferably Anabeana PCC7119. The construct preferably includes a transit peptide to target the protein to the chloroplast. A suitable construct is also shown in FIG. 8. In a preferred embodiment, the construct also comprises a chloroplast targeting sequence, for example a sequence derived from pea. The transit peptide targets the polypeptide to the chloroplast. In preferred embodiments, the construct comprises a sequence as shown in SEQ ID No. 1 or 2. Stable transformants are obtained expressing the FId transgene.

In a second step, a second plant is transformed with a nucleic acid construct comprising a sequence encoding a FNR polypeptide as described herein. Stable transformants that are homozygous for the transgene are generated expressing the FNR transgene.

The nucleic acid construct comprising a nucleic acid sequence encoding a FNR polypeptide and which can be used in the different embodiments of the methods herein is described in detail below. The nucleic acid sequence encoding a FNR is preferably of bacterial origin and most preferably derived from a cyanobacterium.

The cyanobacterium may be a phycobillisome-containing bacterium, for example selected from Crocosphaera, Cyanobium, Cyanothece, Microcystis, Synechococcus, Synechocystis, Thermosynechococcus, Microchaetaceae, Nostocaceae, Lyngbya, Spirulina or Trichodesmium. Preferred genera include Synechococcus, Fremyella, Tolypothrix, Anabaena, Anabaenopsis, Aphanizomenon, Aulosira, Cylindrospermopsis, Cylindrospermum, Loefgrenia, Nodularia, Nostoc or Wollea.

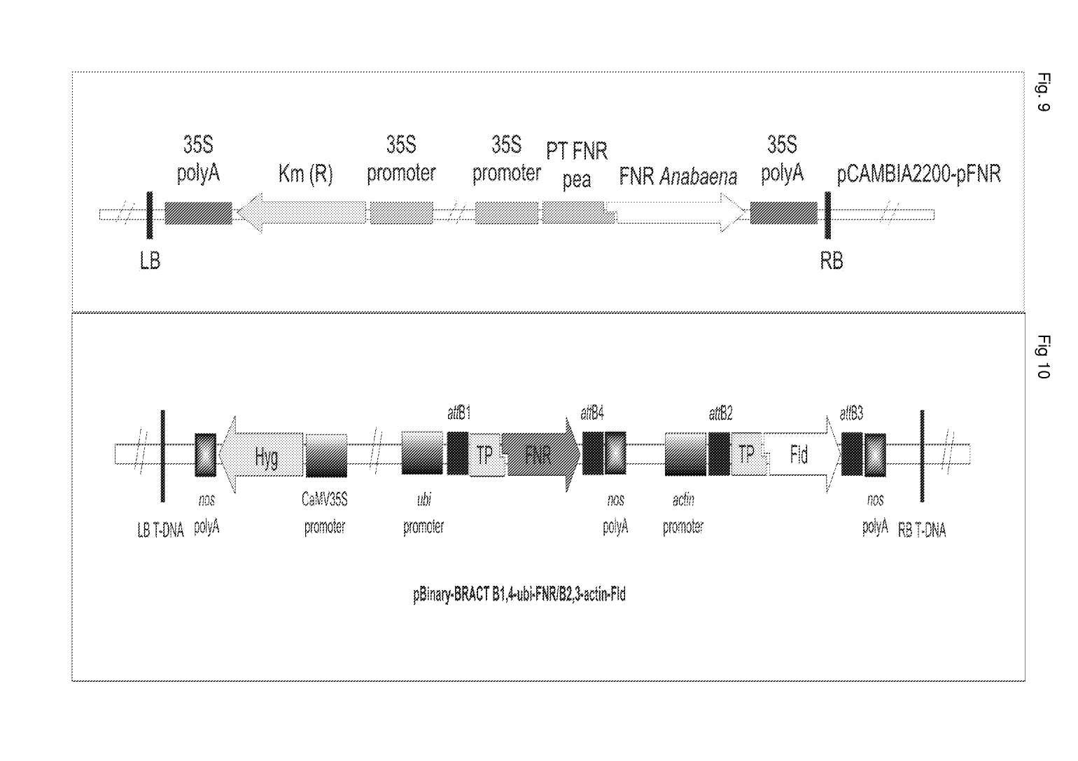

As shown in the examples, the FNR gene from Anabaena PCC7119 can be manipulated. The third domain was deleted and the resulting chimeric gene introduced in tobacco. Thus, in one embodiment, the genus is Anabaena. Preferably, the sequence comprises a sequence encoding the C-terminal two domain region but does not comprise the region encoding the phycobilisome- binding domain. The full length sequence of FNR is shown in SEQ ID NO. 5. For example, the construct may comprise SEQ ID NO. 3. The construct may preferably include a sequence encoding a transit peptide to target the protein to the chloroplast. A transit peptide is a chloroplast targeting peptide. This is preferably derived from a plant FNR, for example pea. For example, the construct may comprise SEQ ID NO. 4. As an example, FIG. 9 shows a construct according to the invention.

In a third step, the stable transformants of the first kind are crossed with stable transformants of the second kind to generate a stable homozygous progeny plant expressing both, FNR and FId. As a skilled person will know, crossing a FId plant and a FNR plant will result in a “hybrid” that is hemizygous for each gene. The resulting plant has to be selfed and then the progeny selected to find double homozygotes—i.e. plants that are homozygous for both transgenes. A skilled person would also know that polyploids require more than one step of “selfing”. Thus, the step of generating a plant homozygous for and expressing both FNR and Fid includes generating progeny of the plants obtained through step d) and selecting a plant that is homozygous for both transgenes. As shown in the examples, after crossing of FNR plants with FId-expressing siblings, double homozygous plants were selected and shown to display greater tolerance to methyl viologen (MV), a redox-cycling compound which causes oxidative stress, relative to single homozygous FId plants.

In another embodiment, the method for producing a plant with enhanced stress tolerance comprises
a) expressing a nucleic acid construct in a plant said construct comprising a sequence encoding a flavodoxin polypeptide or a FNR polypeptide in a plant,
b) transforming said plant with a nucleic acid construct comprising a sequence encoding a flavodoxin polypeptide or a FNR polypeptide respectively to generate a stable homozygous plant expressing FNR and FId.

According to this embodiment, a single transformant is created and the single transformant is transformed again with a nucleic acid construct comprising the second gene to generate a stable homozygous plant expressing FNR and FId. Stable homozygous plants are then selected.

A skilled person will know, that using selective marker genes for the different constructs will help to facilitate selecting double mutants.

The constructs which can be used in this embodiment are also described above.

In another aspect, the invention relates to a nucleic acid construct comprising a nucleic acid sequence encoding a cyanobacterial FNR and a chloroplast targeting sequence. Such constructs and the various embodiments are described above.

In another aspect, the invention relates to a vector comprising a construct as described herein. The vector is preferably suitable for plant transformation and vectors that can be used are known to the skilled person. The invention also relates to a plant host cell comprising a construct or vector as described herein.

The invention also includes host cells containing a recombinant nucleic acid encoding a flavodoxin polypeptide and a recombinant nucleic acid sequence encoding a ferredoxin NADP(H) reductase polypeptide, both as defined hereinabove. Host cells of the invention may be any cell selected from the group consisting of bacterial cells, such as E. coli or Agrobacterium species cells, yeast cells, fungal cells, algal or cyanobacterial cells, or plant cells. In a further embodiment the invention relates to a construct of the invention being comprised in a transgenic plant cell.

In another embodiment the plant cells of the invention are non-propagative cells, e.g. the cells can not be used to regenerate a whole plant from this cell as a whole using standard cell culture techniques, this meaning cell culture methods but excluding in-vitro nuclear, organelle or chromosome transfer methods.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or

(b) genetic control sequence(s) which is operably linked with the nucleic acid sequence according to the invention, for example a promoter, or

(c) a) and b)

are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

Also within the scope of the invention are methods for increasing the stress response or tolerance of a plant comprising expressing a nucleic acid sequence encoding a flavodoxin polypeptide and a nucleic acid sequence encoding a ferredoxin NADP(H) reductase polypeptide in a plant. The method uses the different constructs and steps described herein to produce a stress tolerant plant. Stress response is increased compared to a wild type/control plant and compared to a plant expressing a nucleic acid sequence encoding a flavodoxin polypeptide alone, and not expressing a nucleic acid sequence encoding a ferredoxin NADP(H) reductase polypeptide. Stress response can be increased at least 2 to 10 fold or more.

In another aspect, the invention relates to a transgenic plant obtained or obtainable by a method as described herein. In another aspect, the invention relates to a transgenic plant expressing a construct described herein. The invention also relates to a transgenic plant with increased stress tolerance said transgenic plant expressing a nucleic acid encoding a flavodoxin polypeptide and a nucleic acid encoding ferredoxin NADP(H) reductase polypeptide.

The plant according to the invention expresses a nucleic acid sequence encoding a FNR polypeptide, for example comprising a sequence as shown in SEQ ID No. 8 or 9 or a functional variant thereof, and also expresses a nucleic acid sequence encoding a FId polypeptide, for example comprising a sequence as shown in SEQ ID No. 5, 6 or 7 or a functional variant thereof.

The invention also extends to harvestable parts of a plant such as, but not limited to seeds, leaves, fruits, flowers, stems, roots, rhizomes, tubers, and bulbs, which harvestable parts comprise a recombinant nucleic acid encoding a FNR polypeptide, preferably also comprising a recombinant nucleic acid encoding a flavodoxin polypeptide. The invention furthermore relates to products derived, preferably directly derived, from a harvestable part of such a plant, such as dry pellets or powders, oil, fat and fatty acids, starch or proteins.

The seeds of the invention in one embodiment comprise the constructs of the invention or the vector of the invention. In a further embodiment the seeds of the invention are true-breeding for the construct or the vector of the invention. In another embodiment the seeds contain the a recombinant nucleic acid encoding a FNR polypeptide and also comprise a recombinant nucleic acid encoding a flavodoxin polypeptide, both as disclosed herein, and show increased stress tolerance.

The invention also includes methods for the production of a product comprising a) growing the plants of the invention and b) producing said product from or by the plants of the invention or parts, including seeds, of these plants. In a further embodiment the methods comprises steps a) growing the plants of the invention, b) removing the harvestable parts as defined above from the plants and c) producing said product from or by the harvestable parts of the invention.

The product may be produced at the site where the plant has been grown, or the plants or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the methods of the invention is performed, while allowing repeated times the steps of product production e.g. by repeated removal of harvestable parts of the plants of the invention and if necessary further processing of these parts to arrive at the product. It is also possible that the step of growing the plants of the invention is repeated and plants or harvestable parts are stored until the production of the product is then performed once for the accumulated plants or plant parts. Also, the steps of growing the plants and producing the product may be performed with an overlap in time, even simultaneously to a large extend or sequentially. Generally the plants are grown for some time before the product is produced.

In one embodiment the products produced by said methods of the invention are plant products such as, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. In another embodiment the inventive methods for the production are used to make agricultural products such as, but not limited to, plant extracts, proteins, amino acids, carbohydrates, fats, oils, polymers, vitamins, and the like. It is possible that a plant product consists of one ore more agricultural products to a large extent.

The plant according to the different aspects of the invention may be a monocot or dicot plant. A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, capsicum, tobacco, cotton, oilseed rape, okra, apple, rose, strawberry, alfalfa, bean, soybean, field (fava) bean, pea, lentil, peanut, chickpea, apricots, pears, peach, grape vine or citrus species. In one embodiment, the plant is tobacco. In one embodiment, the plant is barley. In one embodiment, the plant is soybean. In one embodiment, the plant is cotton. In one embodiment, the plant is maize (corn). In one embodiment, the plant is rice. In one embodiment, the plant is oilseed rape including canola. In one embodiment, the plant is wheat. In one embodiment, the plant is sugarcane. In one embodiment, the plant is sugar beet.

Also included are biofuel and bioenergy crops such as rape/canola, linseed, lupin and willow, poplar, poplar hybrids, switchgrass, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant). In another embodiment the invention relates to trees, such as poplar or eucalyptus trees.

A monocot plant may, for example, be selected from the families Arecaceae, Amaryllidaceae or Poaceae. For example, the plant may be a cereal crop, such as wheat, rice, barley, maize, oat, sorghum, rye, onion, leek, millet, buckwheat, turf grass, Italian rye grass, switchgrass, Miscanthus, sugarcane or Festuca species.

Preferably, the plant is a crop plant. By crop plant is meant any plant which is grown on a commercial scale for human or animal consumption or use or other non-food/feed use. Non limiting examples of crop plants include soybean, beet, sugar beet, sunflower, oilseed rape including canola, chicory, carrot, cassava, alfalfa, trefoil, rapeseed, linseed, cotton, tomato, potato, tobacco, poplar, eucalyptus, pine trees, sugarcane and cereals such as rice, maize, wheat, barley, millet, rye, triticale, sorghum, emmer, spelt, secale, einkorn, teff, milo and oats.

Preferred plants are tobacco, maize, wheat, rice, oilseed rape, sorghum, soybean, potato, tomato, barley, pea, bean, cotton, field bean, lettuce, broccoli or other vegetable brassicas or poplar. In another embodiment the plants of the invention and the plants used in the methods of the invention are selected from the group consisting of maize, rice, wheat, soybean, cotton, oilseed rape including canola, sugarcane, sugar beet and alfalfa.

Methods for plant transformation, for example by Agrobacterium mediated transformation or particle bombardment, and subsequent techniques for regeneration and selection of transformed plants are well known in the field. Also within the scope of the invention is chloroplast transformation through biobalistics.

According to the different aspects of the invention, plant stress responses are increased, enhanced or improved. This is understood to mean an increase compared to the level as found in a wild type plant. Moreover, as shown in the examples, the level is also increased with respect to the stress response of a transgenic plant expressing a nucleic sequence encoding FId only. A skilled person will appreciate that such stress responses can be measured and the increase can be 2 to 10 fold. There are two types of stress: biotic stress is imposed by other organisms, such as a pathogen, whereas abiotic stress arises from an excess or deficit in the physical or chemical environment, such as drought, salinity, high or low temperature or high light.

The production and scavenging of chemically reactive species, such as ROS/RNS, are central to a broad range of biotic and abiotic stress and physiological responses in plants. Oxidative stress can be induced by various environmental and biological factors such as hyperoxia, light, drought, high salinity, cold, metal ions, pollutants, xenobiotics, toxins, reoxygenation after anoxia, experimental manipulations, pathogen infection and aging of plant organs.

Thus, the invention relates in particular to methods for increasing or enhancing plant response to oxidative stress, caused for example by extreme temperatures, drought UV light, irradiation, high salinity, cold, metal ions, pollutants, toxins, or pathogen infection by bacteria, viruses or fungi or a combination thereof.

In another embodiment the methods of the invention and plants of the invention relate to enhanced tolerance of stress selected from the group consisting of: drought, low temperature below 15° C. and above freezing point, freezing temperatures, salt stress, nutrient limitation, heavy metal stress, pathogen infection, and combinations thereof.

In another aspect, the invention relates to a method for reducing the amount of ROS in a plant in response to stress comprising expressing a flavodoxin polypeptide and a ferredoxin NADP(H) reductase polypeptide in a plant. According to this method, a construct that directs the expression of both, FId and FNR as described herein may be used. Alternatively, plants expressing FId may be crossed with plants expressing FNR to obtain co-expression of both genes.

In yet another aspect of the present invention methods for increasing the chlorophyll and/ or carotenoid levels of plants or plant parts, e.g. harvestable parts, flowers or seed under stress conditions compared to control plants are claimed.

The relative expression levels of FId and FNR according to the embodiments of the invention may vary with the effect being directly dependent on FId dosis. In a preferred embodiment, the level of expression of FId is at least the same as the expression level of ferredoxin.

### EXAMPLES

The invention will be described in the following non limiting examples.

Methods

Vector Construction

Construction of Ti Vectors for FNR Expression and Co-Expression of FId and FNR in Tobacco

In cyanobacteria, FNR is an intrinsic membrane protein made up of three domains, an FAD binding domain, an NADP(H) binding domain, and an integral domain interacting with phycobilisomes (Fillat et al., 1993), but the first two domains can be separated from the intrinsic domain by either proteolysis or mutagenesis, rendering a soluble two-domain protein which retains full NADPH-ferredoxin (FId) activity (Martínez-Júvez et al., 1996). Such engineering should warrant that cyanobacterial FNR would remain soluble in the chloroplast stroma of the transgenic plants and display only the desired activity. We therefore manipulated the FNR gene from Anabaena PCC7119. The third domain was deleted and the resulting chimeric gene introduced in tobacco. After crossing of FNR plants with FId-expressing siblings, double homozygous plants were selected and shown to display greater tolerance to methyl viologen (MV) toxicity than single homozygous FId plants.

A DNA fragment encoding a region of FNR from Anabaena PCC7119 (without the phycobilisome binding domain) was obtained by PCR amplification of the whole gene cloned into plasmid pTrc99a (Fillat et al., 1990), using primers (primer 1) 5′-CCGAGCTCACACCATGACTCAAGCGAA-3′, (SEQ ID NO 11) and (primer 2) 5′-ACGTCGACCAACTTAGTATGTTTCTAC-3′ (SEQ ID NO 12), complementary to positions 1 to 19 and 906 to 925, respectively. To facilitate further manipulations, a SacI recognition site (GAGCTC) was introduced at the 5′ end of primer 1 and a SalI site (GTCGAC) at the 3′ end of primer 2. PCR conditions were 30 cycles of 60 s at 94° C., 60 s at 54° C. and 90 s at 72° C., using 1 ng of template DNA and 50 pmol of each primer in a medium containing 10 mM Tris-HCl pH 8.4, 5 mM KCl, 1.5 mM MgCl₂, 0.2 mM of each dNTP and 2.5 units of Taq DNA polymerase. After the 30 cycles were completed, the reactions were incubated at 72° C. for 10 min. A purified PCR fragment of the predicted length (940 bp) was digested with Sac! and SalI. The fragment was cloned into compatible sites of a pUC9-derived recombinant plasmid encoding the entire pea FNR precursor (Ceccarelli et al., 1991) between BamHI and SalI restriction sites, and from which the DNA fragment encoding the mature region of pea FNR had been removed by digestion with SacI and SalI. This generated an in-frame fusion of the chloroplast transit peptide derived from pea FNR with the mature region of Anabaena FNR.

The sequence of the chimeric gene was determined on both strands, and excised from the corresponding plasmid by digestion with BamHI and SalI. The 1120-bp fragment was then cloned between the CaMV 35S promoter and polyadenylation regions of pDH51 (Pietrzcak et al., 1986). The entire cassette was further isolated as an EcoRI fragment and inserted into the EcoRI site of the binary vector pCAMBIA 2200 (Hajdukiewiez et al., 1994). The construct was finally mobilised into Agrobacterium tumefaciens strain GV3101 pMP 90 by electroporation (Ausubel et al., 1987).

Construction of Ti Vectors for FNR and FId Expression, and Co-Expression of FId and FNR in Barley

Two independent vectors were developed for using in a co-transformation protocol of barley plants with FNR and FId according to Harwood et al. (2009). All of the molecular biology and recombinant DNA technologies involved are known to the skilled person and explained fully in the literature. The sequence of the chimeric gene comprising the in-frame fusion of the chloroplast transit peptide derived from pea FNR with the C-terminal two-domain encoding region of Anabaena PCC7119 FNR described previously (SEQ ID NO. 4) was amplified by PCR to generate products suitable for cloning in a binary vector of the pBRACT series (Harwood et al, 2009) which contains the hpt gene conferring hygromycin resistance under a 35S promoter at the left border (LB). The chimeric cloned gene is under the control of the maize ubiquitin promoter at the right border (RB). The chimeric construct containing the in-frame fusion of the chloroplast transit peptide derived from pea FNR with the FId coding region of Anabaena PCC7119 (SEQ ID NO. 2, Tognetti et al., 2006; PCT/GB2002/004612), is subjected to a similar protocol as described above.

The resulting binary vectors containing the genes of interest under the control of the desired regulatory sequences may be directly used for plant transformation protocols, for instance Agrobacterium mediated plant tissue transformation or particle bombardment techniques.

Construction of Binary Vectors for the Co-Expression of FId and FNR Polypeptides in Plants

A single construct that can direct the co-expression of FNR and FId polypeptides in a plant transformed with such construct is developed based on the MultiSite Gateway cloning system (Invitrogen, http://www.invitrogen.com) (Karimi et al. 2007; Dafny-Yelin and Tzfira, 2007). FIG. 11 describes the multistep process of design and construction of the above mentioned binary vector. The process is performed following the instructions, protocols and guidelines provided by the manufacturer. All of the molecular biology and recombinant DNA technologies involved are known to the skilled person and explained fully in the literature.

The sequence of the chimeric gene comprising the in-frame fusion of the chloroplast transit peptide derived from pea FNR with the C-terminal two-domain encoding region of Anabaena PCC7119 FNR described previously (SEQ ID NO. 4) is amplified by PCR to generate products suitable for use as substrate in a Gateway BP recombination reaction with an appropriate donor vector. The two gene-specific primers, forward and reverse, are designed in order to incorporate to their 5′ ends the attB1 and attB4 sequences, respectively, required for the specific BP recombination reaction with the attP1 and attP4 sites in the pDONR221 P1-P4 donor vector. The site-specific BP recombination reaction between the attB1-FNR-attB4 PCR product and the pDONR221 P1-P4 vector yields the pENTR221 L1-L4-FNR entry clone, in which the FNR construct is flanked by attL1 and attL4 site-specific sequences for LR recombination. The chimeric construct containing the in-frame fusion of the chloroplast transit peptide derived from pea FNR with the FId coding region of Anabaena PCC7119 (SEQ ID NO. 2, Tognetti et al., 2006; PCT/GB2002/004612), is subjected to a similar protocol as described above, except that the primers incorporate the attB2 and attB3 recombination specific sequences instead of the attB1 and attB4 sites of the former construct. The BP recombination reaction between the resulting attB2-FId-attB3 PCR product and the pDONR221 P2-P3 donor vector yields the pENTR221 L2-L3-FId entry clone in which the FId construct is flanked by the attL2 and attL3 LR recombination specific sites.

The pENTR221 L1-L4-FNR and pENTR221 L2-L3-FId entry clones are used as substrates for a MultiSite Gateway LR recombination reaction with any of the various ad-hoc designed pDEST-BRACT destination vectors (pBRACT). The pDEST-BRACT vectors are MultiSite Gateway destination vectors engineered in order to contain two Gateway cassettes aimed for the independent cloning in a pre-determined orientation of two different constructs flanked by compatible attL sequences by means of a single LR site-specific recombination reaction. They are binary T-DNA vectors containing in addition to the left and right T-DNA border sequences (LB and RB, respectively), a complete plant selection marker expression cassette and plant regulatory regions (promoters, terminators, enhancers) flanking each Gateway cassette to direct the expression of the sequences to be cloned. The various pDEST-BRACT destination vectors developed differ in the identity of the promoters and terminators and/or the attL sequences they contain. They could be customized for optimal expression of the transgenes in monocots or dicots, under the control of constitutive or inducible promoters.

The resulting expression clone is a binary vector containing the genes of interest under the control of the desired regulatory sequences which may be directly used for plant transformation protocols, for instance Agrobacterium mediated plant tissue transformation or particle bombardment techniques.

Expression of FId and FNR in Tobacco

Plant Transformation

Tobacco (Nicotiana tabacum cv Petit Havana) leaf disc transformation was carried out using conventional techniques (Gallois and Marinho, 1995) and the progenies of kanamycin-resistant transformants were analysed further. Primary transformants expressing high levels of cyanobacterial FNR, as evaluated by SDS-PAGE and immunoblotting, were self-pollinated and all subsequent experiments were carried out with the homozygous progeny.

Generation of Transgenic Plants Simultaneously Expressing FId and FNR from Anabaena.

The preparation of double expressing plants was performed by cross-pollination. Transgenic plants expressing FNR from Anabaena (pFNR), generated in this project, and a stable homozygous line expressing high levels of Anabaena FId in chloroplasts (pFId, Tognetti et al., 2006) were used as parentals. Primary double heterozygous transformants expressing pFNR and pFId were self-pollinated and double homozygous plants selected by SDS-PAGE and immunoblotting.

Stress Treatments

Seeds of control and transgenic plants were germinated on Murashige-Skoog (MS) agar supplemented with 3% (w/v) sucrose and, in the case of transformants, 100 μg ml⁻¹ kanamycin. After 4 weeks at 25° C. and 100 μmol quanta m⁻² s⁻¹ (16 h light/8 h dark), plantlets were placed on soil. Leaf discs of 13 mm diameter were punched from young fully expanded leaves of two-month old tobacco plants grown on soil. Discs were weighted and floated individually, top side up, on 1 ml sterile distilled water containing the indicated amounts of MV in 24-well plates, and incubated for 12 h in the dark at 25° C. to allow diffusion of the MV into the leaf. Wells were then illuminated with a white light source at 700 μmol quanta m⁻² s⁻¹. Controls were kept in water under the same conditions. Electrolyte leakage of the leaf discs during MV stress was measured as conductivity of the medium with a Horiba model B-173 conductivity meter. Plantlets grown in soil for 3 or 4 weeks were transferred to a hydroponics system containing Hoagland's solution (Hoagland and Arnon, 1950). After 3 days, the Hoagland's solution was supplemented with 100 μM MV.

Analytical Procedures

Pigment Determination

Chlorophyll and carotenoids contents in leaves and plastids were determined using standard methods (Lichtenthaler, 1987).

Detection of Lipid Peroxides

The FOX assay was used to quantify the presence of lipid peroxides (LOOHs) in plant tissue extracts (DeLong, et al., 2002). Leaf tissue (4 cm²) was extracted with 300 μL of 80:20 (v/v) ethanol:water containing 0.01% butylated hydroxytoluene. Lipids were partitioned into the organic phase, vortexed and centrifuged at 3,000 g. Fifty μl of the plant extract were combined with 50 μl of 10 mM tris-phenylphosphine (TPP, a LOOH reducing agent) in methanol and 500 U bovine liver catalase (Sigma). The mixture was stirred and incubated for 30 min to allow for complete reduction of any present —OOHs by TPP (+TPP). Samples without TPP (−TPP) addition were treated identically except that the TPP aliquot was substituted with methanol. Following the 30 min TPP incubation, 900 μl of a FOX reagent made up of 90% methanol (v/v), 25 mM H₂SO₄, 4 mM butylated hydroxitoluene (BHT), 25 μM of ferrous ammonium sulfate hexahydrate and 100 μM xylenol orange were added to each sample with the absorbance at 560 nm being recorded 10 min after addition in an Ultrospec 1100 spectrophotometer (Amersham, Biosciences). The absorbance difference between the samples without and with TPP indicated the presence of LOOHs; —OOH values were then expressed as micromolar H₂O₂ equivalents using a standard curve spanning a 0-20 μM H₂O₂ range.

Enzyme Activity Assays

For the identification of enzymes displaying NADPH-dependent diaphorase activity, leaf extracts corresponding to 15 μg of soluble protein were resolved by nondenaturing PAGE on 12% polyacrylamide gels. After electrophoresis, the gel was stained by incubation in 50 mM Tris-HCl, pH 8.5, 0.3 mM NADP⁺, 3 mM Glc-6-P, 1 unit ml⁻¹ Glc-6-P dehydrogenase, and 1 mg ml⁻¹ nitroblue tetrazolium until the appearance of the purple formazan bands.

The enzymatic activities of ascorbate peroxidases (APX) were determined in native gel using the method of Mittler and Zilinskas (1993).

Results

Expression of Soluble Anabaena FNR in Transgenic Tobacco Chloroplasts

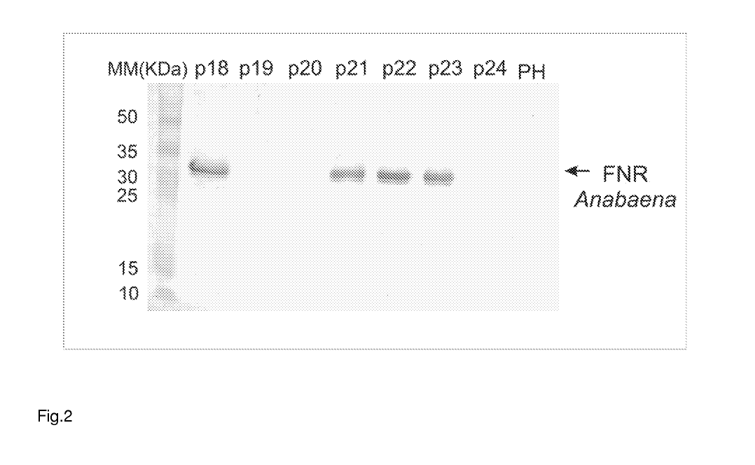

To express a soluble cyanobacterial FNR in tobacco plastids, a chimeric gene was prepared in which the C-terminal, two-domain Anabaena FNR coding region (Fillat et al., 1990) was fused in-frame, at the amino terminus, to a DNA sequence encoding the chloroplast transit peptide of pea FNR (for details, see Methods). The construct was cloned into an Agrobacterium binary vector under the control of the constitutive CaMV 35S gene promoter, and delivered into tobacco cells via Agrobacterium-mediated leaf disc transformation. Kanamycin-resistant plants were recovered from tissue culture and evaluated for FNR accumulation by immunoblotting. Proteins extracted from sampled primary transformants (pFNR) or from a wild-type tobacco specimen (PH) were resolved by SDS-PAGE, and either stained with Coomassie Brilliant Blue, or blotted onto nitrocellulose membranes and probed with antisera raised against Anabaena FNR using standard techniques (FIG. 2).

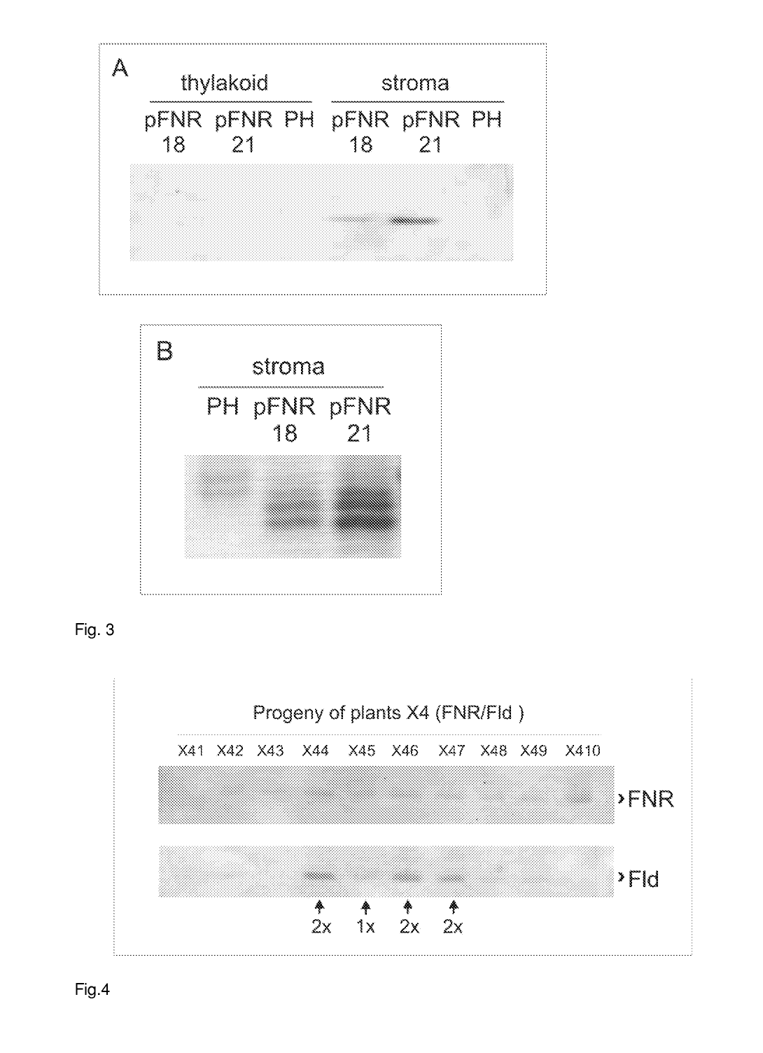

A mature-sized reactive band could be detected at various levels in leaf extracts obtained from several transformants, suggesting plastid import and processing of the expressed flavoprotein. While FNR was detected in the stroma of the chloroplasts of transgenic plants, there was no immunoreactivity in the thylakoid membranes fraction (FIG. 3A). The diaphorase activity of the stromal fraction of the chloroplasts revealed that the enzyme is active in the transgenic tobacco plants (FIG. 3B).

Plants expressing the cyanobacterial FNR in chloroplasts looked phenotypically normal relative to wild-type siblings, and exhibited wild-type levels of tolerance to MV toxicity (data not shown).

Expression of Anabaena FNR and FId in Transgenic Tobacco Chloroplasts.

To obtain double expressing plants, cross-pollination was performed between homozygous plants expressing either FNR or FId. The resulting progeny contained only double heterozygous specimens, as anticipated. They were self-pollinated and double homozygous (2x) plants were selected by Western blot (FIG. 4).

Tolerance to Methyl Viologen

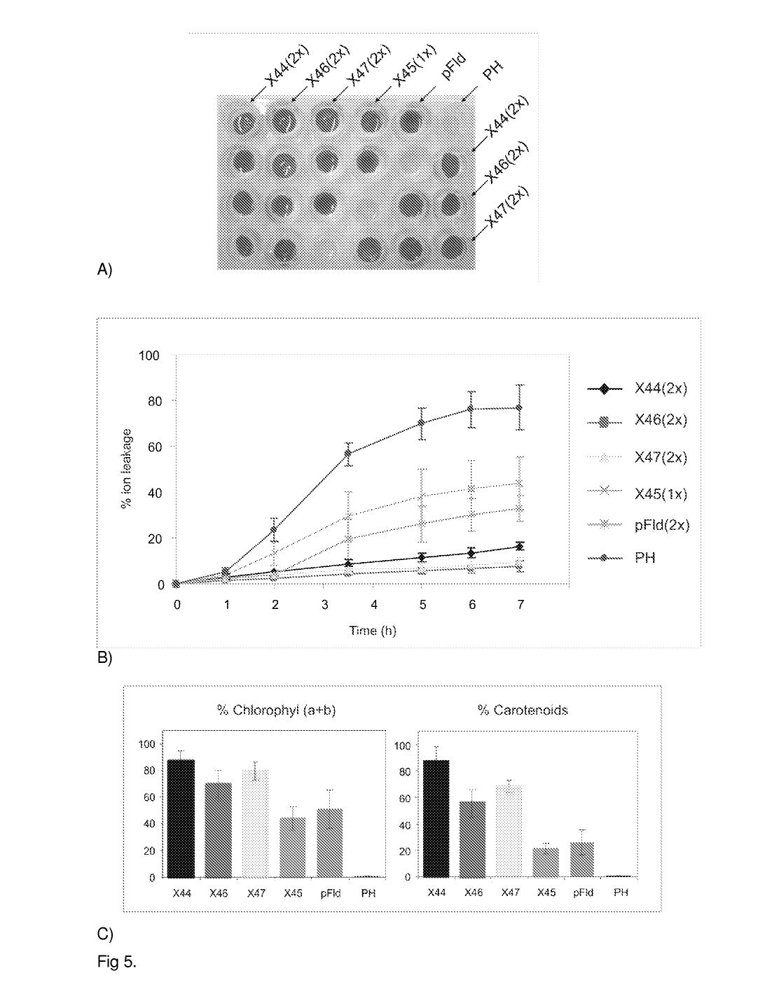

Experiments were performed to evaluate the tolerance of FNR/FId expressing leaf discs to MV as described in Methods. Leaf tissue bleaching was perceived visually in the control discs, reflecting increased chlorophyll degradation (FIG. 5A). Membrane damage due to MV exposure was estimated by measuring electrolyte leakage. Conductance values were corrected for ion leakage occurring in water under the same conditions and expressed as a percentage of the total ion content (maximal value obtained after autoclaving the leaf disks at the end of the MV treatment). Chlorophyll contents were expressed as the fraction of the total chlorophyll of leaf disks incubated under the same conditions in the absence of MV. Both membrane deterioration and pigment integrity were significantly more preserved in double homozygous FNR/FId plants than in single homozygous FId-expressing siblings (FIGS. 5B, C).

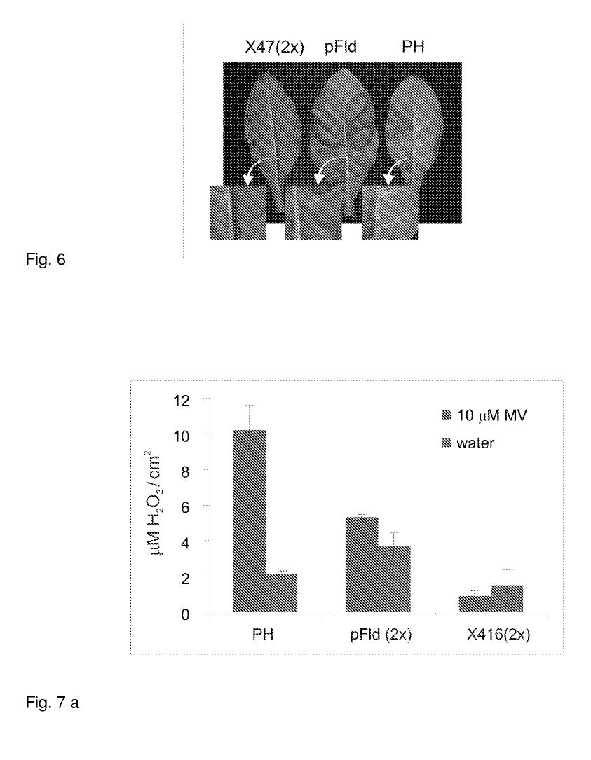

To evaluate the tolerance to MV of whole plants, they were assayed in a hydroponics system as described in Methods. The simultaneous expression of FNR and FId provided more protection against MV-induced damage than the expression of FId alone (FIG. 6).

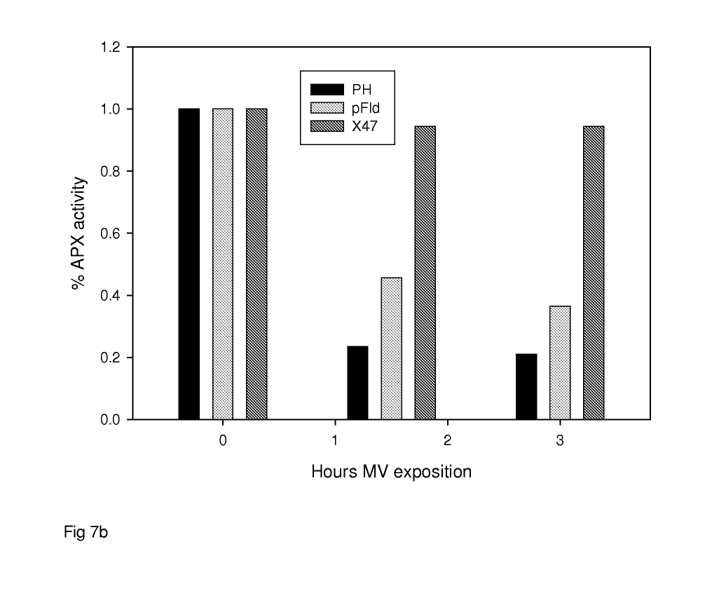

To evaluate ROS propagation, lipid peroxidation was measured by the FOX assay (Delong et al., 2002). Leaf discs of wild-type and transgenic tobacco plants were treated with 10 μM MV as described in Methods. Levels of lipid hydroperoxides (LOOHs) were expressed in μM H₂O₂ cm⁻², and were significantly lower in the double homozygous cross X416 than the homozygous parental pFId. Both were more tolerant than wild-type plants (FIG. 7A). Several proteins are also preferred targets of ROS. Chloroplast ascorbate peroxidase (APX) is one of the most sensitive among them. Exposure of wild-type plants to 20 μM MV leads to 70-80% decline in the activity of this enzyme after only 90 min of incubation. Expression of FId provides partial protection (40-50% of residual activity). The simultaneous presence of FNR in FId-expressing plants leads to almost quantitative preservation of APX activity (FIG. 7B).

Expression and Co-Expression of FId and FNR in Barley

Plant Transformation. Generation of Transgenic Barley Plants Simultaneously Expressing FId and FNR from Anabaena.

Barley was transformed using pBract214 vectors comprising FId and FNR genes, respectively, as described above. The vectors were transformed independently into Agrobacterium tumefaciens and spring barley variety Golden Promise was transformed with a mixture of the two Agrobacterium lines. Barley transformation was performed based on the infection of immature embryos with A. tumefaciens followed by the selection of the transgenic tissue on media containing the antibiotic hygromycin. The method lead to the production of fertile independent transgenic lines (Harwood et al, 2009) and the progenies of hygromycin-resistant transformants were analysed further. Primary heterozygous transformants expressing cyanobacterial FNR and FId, as evaluated by SDS-PAGE and immunoblotting, were used for further experiments. In a modification of the protocol described herein, infection of the embryos was carried out with only each of the A. tumefaciens lines carrying one of the cyanobacterial gene constructs to obtain independent heterozygous transgenic lines expressing the FNR or FId constructs.

Stress Treatments in Barley

Transgenic and control barley plants were grown under controlled environment conditions with 15° C. day and 12° C. night temperatures, 80% humidity, with 16 h photoperiod provided by metal halide bulbs (HQI) supplemented with tungsten bulbs at an intensity of 500 μmol quanta m⁻² s⁻¹ at the mature plant canopy level. The soil mix used was composed of Levington M3 compost/Perlite/Grit mixed in a ratio of 2:2:1. Leaf strips of 10-15 mm length were cut from leaves of 6-week old barley plants grown in soil. Leaf strips were then incubated in distilled water containing the indicated amount of MV and 0.05% Tween-20 for 30 minutes at 20° C. in the dark to allow diffusion of the MV into the tissue. The strips were then placed with the adaxial side up in plastic trays and incubated for the indicated time period under a 450 μmol quanta m⁻² s⁻¹ light source. Controls were kept in distilled water containing 0.05% Tween-20. Chlorophyll and carotenoid contents were then estimated as described in 5.1.

Results

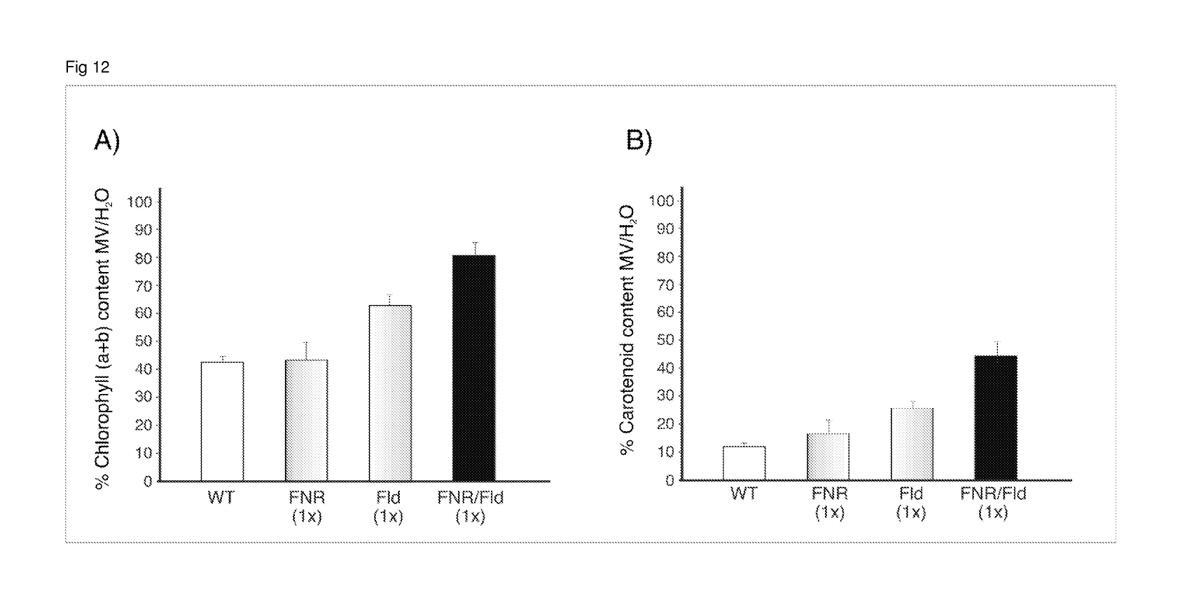

The independent heterozygous barley plants expressing FId and FNR obtained according to the methods described herein were subjected to oxidative stress conditions to evaluate their relative tolerance in comparison to their wild type counterparts. FIG. 12 exhibits typical results obtained when leaf stripes of transgenic plants heterozygous for the FNR and FId genes and wild-type individuals were exposed to the redox cycling herbicide methyl viologen and the content of the photosynthetic pigments chlorophylls and carotenoids were then estimated as described in methods. Pigment degradation is a marker of deterioration of the photosynthetic apparatus. The results show that double heterozygous FNR/FId transgenic plants managed to withstand the oxidative challenge conserving 2- and 4-times higher levels of total chlorophyll and carotenoids, respectively, than the wild-type (and FNR alone) counterparts. FId-expressing transgenic barley plants show an intermediate level of tolerance. The fact that heterozygous plants for both transgenes, FNR and FId, exhibit high levels of tolerance is remarkable given the fact of the dosage dependency of the protective effect conferred by the transgenes.

Concluding Remarks

Simultaneous expression of both FId and FNR from the same cyanobacterial species in plants confers increased tolerance to MV toxicity relative to plants expressing FId alone. For the sake of simplicity, pn plants represent primary FNR tobacco transformants, Xn plants are the crosses of pn plants with pfld5-8 from Tognetti et al. (2006). Xnn or Xnnn are the segregants of self-pollination of X4 double heterozygous plants.

### REFERENCES

Ausubel F. M. et al (1987) Current Protocols in Molecular Cloning. New York, N.Y.: John Wiley and Sons.

Blaschkowski H. P., Neuer G., Ludwig-Fest, M. and Knappe, J. (1982). Eur J Biochem 123, 563-569.

Carrillo N. and Ceccarelli E.A. (2003) Eur. J. Biochem. 270, 1900-1915.

Ceccarelli E. A., A. M. Viale, A. R. Krapp, and N. Carrillo. (1991) J. Biol. Chem. 266, 14283-14287.

Dafny-Yelin M. and Tzfira T. (2007) Delivery of multiple transgenes to plant cells. Plant Physiol., 145, 1118-1128.

DeLong J. M., Prange R. K., Hodges D. M., Forney C. F., Bishop M. C. and Quilliam M. (2002). J. Agric. Food. Chem. 50, 248-254.

Dezar, C A, Fedrigo, G V, Chan, R L. (2005) Plant Science 169, 447-456

Fillat M F, Bakker H A, Weisbeek P J. (1990) Nucleic Acids Res., 18, 7161.

Fillat M. F., Flores, E. and Gomez-Moreno, C. (1993), Plant Mol. Biol. 22, 725-729.

Forti G. and Bracale M. (1984) FEBS Lett 166, 81-84.

Gallois P. and Marinho P. (1995) In H Jones, ed, Methods in Molecular Biology, Vol 49. Humana Press, Totowa, N.J., pp 39-48

Hajdukiewiez P., Svab Z. and Maliga P. (1994) Plant Mol. Biol. 25, 989-994

Harwood W A, Bartlett J G, Alves S C, Perry M, Smedley M A, Leyland N, Snape J W (2009) Barley transformation using Agrobacterium-mediated techniques. Methods Mol Biol 478: 137-147

Karimi M., Bleys A., Vanderhaeghen R. and Hilson P. (2007) Building blocks for plant gene assembly. Plant Physiol., 145, 1183-1191.

Kim et al (2008) No single way to understand singlet oxygen signalling in plants EMBO Rep. 2008 May; 9(5): 435-439.

Lichtenthaler H. K. (1987) Methods Enzymol. 148, 350-382.

Martinez-Júlvez M., Hurley J. K., Tollin G., Gomez-Moreno C., Fillat M. (1996) Biochim. Biophys. Acta 1297, 200-206.

Mittler, R, Zilinskas B. A. (1993) Detection of ascorbate peroxidase activity in native gels by inhibition of the ascorbate-dependent reduction of nitroblue tetrazolium. Anal Biochem 212, 540-546.

Pastori Gabriela M. (2002) Common Components, Networks, and Pathways of Cross-Tolerance to Stress. The Central Role of “Redox” and Abscisic Acid-Mediated Controls Plant Physiol, June 2002, Vol. 129, pp. 460-468

Pietrzcak M, Shillito R M, Hohn T and Potrikus I (1986) Nucleic Acids Res 14, 5857-5868.

Razquin P., Schmitz S., Peleato M. L., Fillat M. F., Gómez-Moreno C. and Böhme H. (1995). Photosynthesis Research 43, 35-40.

Sancho J., Peleato M. L., Golmez-Moreno C. and Edmondson D. E. (1987) Arch. Biochem. Biophys. 288, 231-238

Sandmann G., Peleato M. L., Fillat M. F., Lázaro M. C. and Gomez-Moreno C. (1990). Photosynthesis Research 26, 119-125.

Schluchter W. M. and Bryant, D. A. (1992) Biochemistry 31, 3092-3102

Singh, A. K., Li, H. and Sherman, L. A. (2004) Microarray analysis and redox control of gene expression in the cyanobacterium Synechocystis sp. PCC6803. Physiol. Plant., 120, 27-35.

Thomas J. C., Ughy B., Lagoutte B. and Ajani G. (2006) Proc. Natl. Acad. Sci. USA, 103, 18368-18373.

Tognetti V. B., Palatnik, J. F., Fillat M. F., Melzer M., Hajirezaei M.-R., Valle E. M. and Carrillo, N. (2006) Functional replacement of ferredoxin by a cyanobacterial flavodoxin in tobacco confers broad-range stress tolerance. Plant Cell, 18, 2035-2050.

Tognetti V. B., Monti, M. R., Valle E. M., Carrillo N. and Smania, A. M. (2007a) Detoxification of 2,4-dinitrotoluene by transgenic tobacco plants expressing a bacterial flavodoxin. Environ. Sci. Technol., 41, 4071-4076.

Tognetti V. B., Zurbriggen M. D., Morandi E. N., Fillat, M. F., Valle E. M., Hajirezaei, M.-R. and Carrillo, N. (2007b) Enhanced plant tolerance to iron starvation by functional substitution of chloroplast ferredoxin with a bacterial flavodoxin. Proc. Natl. Acad. Sci. USA, 104, 11495-11500.

Vranová et al (2002) Signal transduction during oxidative stress. J Exp Bot. 2002 May; 53(372):1227-36

Zurbriggen M. D., Tognetti, V. B. and Carrillo N. (2007) Stress-inducible flavodoxin from photosynthetic microorganisms. The mystery of flavodoxin loss from the plant genome. IUBMB Life, 59, 355-360.

Sequence Listing

Nucleic acid sequences as described herein and corresponding peptides are listed below.

Seq 1:
Fld nucleic acid sequence for use in single
fusion construct without targeting sequence
ATGTCAAAGAAAATTGGTTTATTCTACGGTACTCAAACTGGTAAAACTGAATCAGT
AGCAGAAATCATTCGAGACGAGTTTGGTAATGATGTGGTGACATTACACGATGTTT
CCCAGGCAGAAGTAACTGACTTGAATGATTATCAATATTTGATTATTGGCTGTCCT
ACTTGGAATATTGGCGAACTGCAAAGCGATTGGGAAGGACTCTATTCAGAACTGG
ATGATGTAGATTTTAATGGTAAATTGGTTGCCTACTTTGGGACTGGTGACCAAATA
GGTTACGCAGATAATTTTCAGGATGCGATCGGTATTTTGGAAGAAAAAATTTCTCA
ACGTGGTGGTAAAACTGTCGGCTATTGGTCAACTGATGGATATGATTTTAATGATT
CCAAGGCACTAAGAAATGGCAAGTTTGTAGGACTAGCTCTTGATGAAGATAATCAA
TCTGACTTAACAGACGATCGCATCAAAAGTTGGGTTGCTCAATTAAAGTCTGAATT
TGGTTTGTAA
Seq 2:
Fld nucleic acid sequence for use in single
fusion construct with targeting sequence
ATGGCTGCTGCAGTAACAGCCGCAGTCTCCTTGCCATACTCCAACTCCACTTCCC
TTCCGATCAGAACATCTATTGTTGCACCAGAGAGACTTGTCTTCAAAAAGGTTTCA
TTGAACAATGTTTCTATAAGTGGAAGGGTAGGCACCATCAGAGCTCTCATAATGTC
AAAGAAAATTGGTTTATTCTACGGTACTCAAACTGGTAAAACTGAATCAGTAGCAG
AAATCATTCGAGACGAGTTTGGTAATGATGTGGTGACATTACACGATGTTTCCCAG
GCAGAAGTAACTGACTTGAATGATTATCAATATTTGATTATTGGCTGTCCTACTTGG
AATATTGGCGAACTGCAAAGCGATTGGGAAGGACTCTATTCAGAACTGGATGATG
TAGATTTTAATGGTAAATTGGTTGCCTACTTTGGGACTGGTGACCAAATAGGTTAC
GCAGATAATTTTCAGGATGCGATCGGTATTTTGGAAGAAAAAATTTCTCAACGTGG
TGGTAAAACTGTCGGCTATTGGTCAACTGATGGATATGATTTTAATGATTCCAAGG
CACTAAGAAATGGCAAGTTTGTAGGACTAGCTCTTGATGAAGATAATCAATCTGAC
TTAACAGACGATCGCATCAAAAGTTGGGTTGCTCAATTAAAGTCTGAATTTGGTTT
GTAA
Seq 3
FNR Anabaena PCC7119 nucleic acid sequence
for use in single fusion construct (sequence
encoding two domains) without targeting sequence
ATGACTCAAGCGAAAGCCAAACACGCTGATGTTCCTGTTAATCTTTACCGTCCCAA
TGCTCCATTTATTGGTAAGGTAATCTCTAATGAACCACTGGTAAAAGAAGGCGGGA
TAGGTATTGTTCAGCACATTAAATTTGATCTAACTGGTGGTAACTTAAAGTACATCG
AAGGTCAAAGTATTGGTATCATTCCACCAGGAGTGGACAAGAACGGCAAGCCGGA
AAAATTGAGACTCTACTCCATTGCCTCGACCCGTCACGGCGATGATGTGGATGAT
AAAACCATCTCACTGTGCGTCCGTCAATTAGAGTACAAACATCCAGAAAGCGGCG
AAACAGTTTACGGTGTTTGTTCTACTTACTTGACTCACATTGAACCAGGTTCAGAA
GTGAAAATCACTGGGCCTGTGGGTAAAGAAATGCTGTTACCCGATGATCCTGAAG
CTAATGTCATCATGTTGGCAACAGGTACTGGTATTGCGCCTATGCGGACTTACCT
GTGGCGGATGTTCAAGGATGCAGAAAGAGCTGCTGACCCAGAATATCAATTCAAA
GGATTCTCTTGGTTAGTCTTTGGTGTTCCTACAACTCCTAACATTCTTTATAAAGAA
GAACTGGAAGAAATCCAACAAAAATATCCCGATAACTTCCGCCTAACTTACGCTAT
CAGCCGGGAGCAAAAGAATCCCCAAGGTGGCAGAGTGTACATCCAAGACCGTGT
GGCAGAACACGCTGATGAACTGTGGCAATTAATCAAGAATGAAAAAACCCACACC
TACATCTGTGGTTTGCGCGGTATGGAAGAGGGCATTGATGCTGCTTTAAGTGCTG
CGGCTGCGAAAGAAGGTGTTACCTGGAGTGATTACCAAAAAGACCTCAAGAAAGC
TGGTCGCTGGCACGTAGAAACATACTAA
Seq 4
FNR nucleic acid sequence for use in single
fusion construct (FNR construct with sequence
encoding two domains) with targeting sequence
ATGGCTGCTGCAGTAACAGCCGCAGTCTCCTTGCCATACTCCAACTCCACTTCCC
TTCCGATCAGAACATCTATTGTTGCACCAGAGAGACTTGTCTTCAAAAAGGTTTCA
TTGAACAATGTTTCTATAAGTGGAAGGGTAGGCACCATCAGAGCTCACACCATGA
CTCAAGCGAAAGCCAAACACGCTGATGTTCCTGTTAATCTTTACCGTCCCAATGCT
CCATTTATTGGTAAGGTAATCTCTAATGAACCACTGGTAAAAGAAGGCGGGATAG
GTATTGTTCAGCACATTAAATTTGATCTAACTGGTGGTAACTTAAAGTACATCGAAG
GTCAAAGTATTGGTATCATTCCACCAGGAGTGGACAAGAACGGCAAGCCGGAAAA
ATTGAGACTCTACTCCATTGCCTCGACCCGTCACGGCGATGATGTGGATGATAAA
ACCATCTCACTGTGCGTCCGTCAATTAGAGTACAAACATCCAGAAAGCGGCGAAA
CAGTTTACGGTGTTTGTTCTACTTACTTGACTCACATTGAACCAGGTTCAGAAGTG
AAAATCACTGGGCCTGTGGGTAAAGAAATGCTGTTACCCGATGATCCTGAAGCTA
ATGTCATCATGTTGGCAACAGGTACTGGTATTGCGCCTATGCGGACTTACCTGTG
GCGGATGTTCAAGGATGCAGAAAGAGCTGCTGACCCAGAATATCAATTCAAAGGA
TTCTCTTGGTTAGTCTTTGGTGTTCCTACAACTCCTAACATTCTTTATAAAGAAGAA
CTGGAAGAAATCCAACAAAAATATCCCGATAACTTCCGCCTAACTTACGCTATCAG
CCGGGAGCAAAAGAATCCCCAAGGTGGCAGAGTGTACATCCAAGACCGTGTGGC
AGAACACGCTGATGAACTGTGGCAATTAATCAAGAATGAAAAAACCCACACCTACA
TCTGTGGTTTGCGCGGTATGGAAGAGGGCATTGATGCTGCTTTAAGTGCTGCGGC
TGCGAAAGAAGGTGTTACCTGGAGTGATTACCAAAAAGACCTCAAGAAAGCTGGT
CGCTGGCACGTAGAAACATACTAA
Seq 5:
FNR full nucleic acid sequence (with 3 domains)
ATGTCTAATCAAGGTGCTTTTGATGGTGCTGCCAACGTAGAATCAGGTAGCCGCG
TCTTCGTTTACGAAGTGGTGGGTATGCGTCAGAACGAAGAAACTGATCAAACGAA
CTACCCAATTCGTAAAAGTGGCAGTGTGTTCATTAGAGTGCCTTACAACCGCATGA
ATCAAGAAATGCAGCGTATCACTCGACTAGGCGGCAAGATTGTTACGATTCAAAC
AGTAAGCGCACTACAACAACTCAATGGTAGAACTACCATTGCAACAGTAACAGATG
CGTCTAGTGAGATTGCTAAGTCTGAGGGGAATGGTAAAGCCACACCTGTAAAAAC
TGATAGTGGAGCTAAAGCGTTCGCTAAACCACCAGCTGAAGAACAGCTTAAGAAA
AAAGACAACAAAGGCAACACCATGACTCAAGCGAAAGCCAAACACGCTGATGTTC
CTGTTAATCTTTACCGTCCCAATGCTCCATTTATTGGTAAGGTAATCTCTAATGAAC
CACTGGTAAAAGAAGGCGGGATAGGTATTGTTCAGCACATTAAATTTGATCTAACT
GGTGGTAACTTAAAGTACATCGAAGGTCAAAGTATTGGTATCATTCCACCAGGAGT
GGACAAGAACGGCAAGCCGGAAAAATTGAGACTCTACTCCATTGCCTCGACCCGT
CACGGCGATGATGTGGATGATAAAACCATCTCACTGTGCGTCCGTCAATTAGAGT
ACAAACATCCAGAAAGCGGCGAAACAGTTTACGGTGTTTGTTCTACTTACTTGACT
CACATTGAACCAGGTTCAGAAGTGAAAATCACTGGGCCTGTGGGTAAAGAAATGC
TGTTACCCGATGATCCTGAAGCTAATGTCATCATGTTGGCAACAGGTACTGGTATT
GCGCCTATGCGGACTTACCTGTGGCGGATGTTCAAGGATGCAGAAAGAGCTGCT
GACCCAGAATATCAATTCAAAGGATTCTCTTGGTTAGTCTTTGGTGTTCCTACAAC
TCCTAACATTCTTTATAAAGAAGAACTGGAAGAAATCCAACAAAAATATCCCGATAA
CTTCCGCCTAACTTACGCTATCAGCCGGGAGCAAAAGAATCCCCAAGGTGGCAGA
GTGTACATCCAAGACCGTGTGGCAGAACACGCTGATGAACTGTGGCAATTAATCA
AGAATGAAAAAACCCACACCTACATCTGTGGTTTGCGCGGTATGGAAGAGGGCAT
TGATGCTGCTTTAAGTGCTGCGGCTGCGAAAGAAGGTGTTACCTGGAGTGATTAC
CAAAAAGACCTCAAGAAAGCTGGTCGCTGGCACGTAGAAACATACTAA
SEQ 6:
Fld amino acid sequence
MSKKIGLFYGTQTGKTESVAEIIRDEFGNDVVTLHDVSQAEVTDLNDYQYLIIGCPTWN
IGELQSDWEGLYSELDDVDFNGKLVAYFGTGDQIGYADNFQDAIGILEEKISQRGGKT
VGYWSTDGYDFNDSKALRNGKFVGLALDEDNQSDLTDDRIKSWVAQLKSEFGL
SEQ 7::
Fld amino acid sequence with targeting sequence
MAAAVTAAVSLPYSNSTSLPIRTSIVAPERLVFKKVSLNNVSISGRVGTIRALIMSKKIGL
FYGTQTGKTESVAEIIRDEFGNDVVTLHDVSQAEVTDLNDYQYLIIGCPTWNIGELQSD
WEGLYSELDDVDFNGKLVAYFGTGDQIGYADNFQDAIGILEEKISQRGGKTVGYWST
DGYDFNDSKALRNGKFVGLALDEDNQSDLTDDRIKSWVAQLKSEFGL
Seq 8:
FNR Anabaena PCC7119 amino acid sequence (2 domain)
without targeting sequence
MTQAKAKHADVPVNLYRPNAPFIGKVISNEPLVKEGGIGIVQHIKFDLTGGNLKYIEGQ
SIGIIPPGVDKNGKPEKLRLYSIASTRHGDDVDDKTISLCVRQLEYKHPESGETVYGVC
STYLTHIEPGSEVKITGPVGKEMLLPDDPEANVIMLATGTGIAPMRTYLWRMFKDAER
AADPEYQFKGFSWLVFGVPTTPNILYKEELEEIQQKYPDNFRLTYAISREQKNPQGGR
VYIQDRVAEHADELWQLIKNEKTHTYICGLRGMEEGIDAALSAAAAKEGVTWSDYQKD
LKKAGRWHVETY
Seq 9:
FNR Anabaena PCC7119 amino acid sequence (2 domain)
with targeting sequence
MAAAVTAAVSLPYSNSTSLPIRTSIVAPERLVFKKVSLNNVSISGRVGTIRAHTMTQAK
AKHADVPVNLYRPNAPFIGKVISNEPLVKEGGIGIVQHIKFDLTGGNLKYIEGQSIGIIPP
GVDKNGKPEKLRLYSIASTRHGDDVDDKTISLCVRQLEYKHPESGETVYGVCSTYLTH
IEPGSEVKITGPVGKEMLLPDDPEANVIMLATGTGIAPMRTYLWRMFKDAERAADPEY
QFKGFSWLVFGVPTTPNILYKEELEEIQQKYPDNFRLTYAISREQKNPQGGRVYIQDR
VAEHADELWQLIKNEKTHTYICGLRGMEEGIDAALSAAAAKEGVTWSDYQKDLKKAG
RWHVETY
Seq 10:
FNR full amino acid sequence (3 domain sequence)
without targeting sequence
MSNQGAFDGAANVESGSRVFVYEVVGMRQNEETDQTNYPIRKSGSVFIRVPYNRMN
QEMQRITRLGGKIVTIQTVSALQQLNGRTTIATVTDASSEIAKSEGNGKATPVKTDSGA
KAFAKPPAEEQLKKKDNKGNTMTQAKAKHADVPVNLYRPNAPFIGKVISNEPLVKEG
GIGIVQHIKFDLTGGNLKYIEGQSIGIIPPGVDKNGKPEKLRLYSIASTRHGDDVDDKTIS
LCVRQLEYKHPESGETVYGVCSTYLTHIEPGSEVKITGPVGKEMLLPDDPEANVIMLA
TGTGIAPMRTYLWRMFKDAERAADPEYQFKGFSWLVFGVPTTPNILYKEELEEIQQKY
PDNFRLTYAISREQKNPQGGRVYIQDRVAEHADELWQLIKNEKTHTYICGLRGMEEGI
DAALSAAAAKEGVTWSDYQKDLKKAGRWHVETY

1.-52. (canceled)

53. A method for producing a plant with enhanced stress tolerance comprising expressing a nucleic acid sequence encoding a flavodoxin polypeptide (Fld) and a nucleic acid sequence encoding a ferredoxin NADP(H) reductase polypeptide (FNR) in a plant, wherein said nucleic acid sequences are of bacterial origin.

54. A method according to claim 53, comprising expressing a nucleic acid construct in said plant wherein said nucleic acid construct comprises a nucleic acid sequence encoding a flavodoxin polypeptide and a nucleic acid sequence encoding a ferredoxin NADP(H) reductase polypeptide.

55. A method according to claim 54, wherein said construct directs the co-expression of a flavodoxin and a ferredoxin NADP(H) reductase polypeptide.

56. A method according to claim 53, comprising
a) expressing a nucleic acid construct in a first plant, said construct comprising a sequence encoding a flavodoxin polypeptide,
b) expressing a nucleic acid construct in a second plant, said construct comprising a nucleic acid sequence encoding a FNR polypeptide,
c) crossing the first and second plant, and
d) generating a stable homozygous plant expressing FNR and Fld.

57. A method according to claim 53, comprising
a) expressing a nucleic acid construct in a plant, said construct comprising a sequence encoding a flavodoxin polypeptide or a FNR polypeptide,
b) transforming said plant with a nucleic acid construct comprising a sequence encoding a flavodoxin polypeptide or a sequence encoding a FNR polypeptide, respectively, to generate a stable homozygous plant expressing FNR and Fld.

58. A method according to claim 53, wherein the nucleic acid sequence encoding a flavodoxin polypeptide is (a) derived from a cyanobacterium and the flavodoxin polypeptide is a cyanobacterial flavodoxin or (b) derived from a heterotrophic bacterium.

59. A method according to claim 53, wherein the nucleic acid sequence encoding a flavodoxin polypeptide is selected from the group consisting of the nucleic acid sequences shown in Table 1.

60. A method according to claim 53, wherein the nucleic acid sequence encoding ferredoxin NADP(H) reductase is derived from a cyanobacterium polypeptide and the ferredoxin NADP(H) reductase polypeptide is a cyanobacterial FNR.

61. A method according to claim 53, wherein the nucleic acid sequence encoding a ferredoxin NADP(H) reductase polypeptide is selected from the group consisting of nucleic acid sequences shown in Table 2.

62. A method according to claim 58, wherein the cyanobacterium is selected from Crocosphaera, Cyanobium, Cyanothece, Microcystis, Synechococcus, Synechocystis, Thermosynechococcus, Microchaetaceae, Nostocaceae, Lyngbya, Spirulina or Trichodesmium.

63. A method according to claim 60, wherein the cyanobacterium is selected from Crocosphaera, Cyanobium, Cyanothece, Microcystis, Synechococcus, Synechocystis, Therm osynechococcus, Microchaetaceae, Nostocaceae, Lyngbya, Spirulina or Trichodesmium.

64. A method according to claim 58, wherein the cyanobacterium is selected from Fremyella, Tolypothrix, Anabaena, Anabaenopsis, Aphanizomenon, Aulosira, Cylindrospermopsis, Cylindrospermum, Loefgrenia, Nodularia, Nostoc or Wollea.

65. A method according to claim 60, wherein the cyanobacterium is selected from Fremyella, Tolypothrix, Anabaena, Anabaenopsis, Aphanizomenon, Aulosira, Cylindrospermopsis, Cylindrospermum, Loefgrenia, Nodularia, Nostoc or Wollea.

66. A method according to claim 53, wherein the nucleic acid sequence encoding a cyanobacterial flavodoxin comprises SEQ ID NO. 1 or a functional variant thereof.

67. A method according to claim 53 wherein said nucleic acid sequence encoding ferredoxin NADP(H) reductase comprises a sequence encoding the C-terminal two domain region but does not comprise a sequence encoding the phycobillisome-binding domain.

68. A method according to claim 67, wherein the nucleic acid sequence encoding a cyanobacterial FNR comprises SEQ ID NO. 3 or a functional variant thereof.

69. A method according to claim 54, wherein said nucleic acid construct further comprises a regulatory sequence.

70. A method according to claim 54, wherein said construct further comprises a chloroplast targeting sequence.

71. A method according to claim 70, wherein said chloroplast targeting sequence is derived from pea FNR.

72. A method according to claim 71, wherein the nucleic acid sequence encoding a flavodoxin polypeptide comprises SEQ ID NO. 2 or a functional variant thereof.

73. A method according to claim 72, wherein the nucleic acid sequence encoding a FNR polypeptide comprises SEQ ID NO. 4 or a functional variant thereof.

74. A method according to claim 53, wherein said plant is a monocot or dicot plant.

75. A method according to claim 74, wherein said plant is a crop plant.

76. A method according to claim 75, wherein said plant is tobacco or barley.

77. A method according to claim 53, wherein said stress is selected from biotic or abiotic stress.

78. A method according to claim 77, wherein said stress is selected from UV light, extreme temperatures, water deficiency, salinity, drought, and pathogen infection.

79. A transgenic plant produced by the method of claim 53.

80. A transgenic plant with increased stress tolerance, said transgenic plant expressing a nucleic acid encoding a flavodoxin polypeptide and a nucleic acid encoding ferredoxin NADP(H) reductase polypeptide wherein said nucleic acid sequences are of bacterial origin.

81. A plant according to claim 80, wherein said plant is a monocot or dicot plant.

82. A transgenic plant according to claim 81, wherein said plant is a crop plant.

83. A plant according to claim 85, wherein said plant is tobacco or barley.

84. A plant according to claim 80, wherein said plant expresses nucleotide sequence SEQ ID Nos. 2 and 4 or a functional variant of one or both thereof.

85. A nucleic acid construct comprising a nucleic acid sequence encoding a flavodoxin polypeptide and a nucleic acid sequence encoding a ferredoxin NADP(H) reductase polypeptide, wherein said nucleic acid sequences are of bacterial origin.

86. A construct according to claim 83 wherein said bacterium is a cyanobacterium.

87. A construct according to claim 85, wherein said construct further comprises a chloroplast targeting sequence.

88. A construct according to claim 87, wherein said chloroplast targeting sequence is derived from pea FNR.

89. A construct according to claim 88, wherein the nucleic acid sequence encoding a flavodoxin polypeptide comprises SEQ ID NO. 2 or a functional variant thereof.

90. A construct according to claim 88, wherein the nucleic acid sequence encoding a FNR polypeptide comprises SEQ ID NO. 4 or a functional variant thereof.

91. A vector comprising a construct according to claim 85.

92. A host cell comprising a construct according to a vector of claim 91.

93. A plant cell comprising a construct according to claim 85.

94. A transgenic plant, plant part including seed or plant cell, wherein said plant or part thereof comprises a recombinant nucleic acid construct according to claim 85.

95. A transgenic plant according to claim 94, wherein said plant is a monocot or dicot plant.

96. A transgenic plant according to claim 95, wherein said plant is a crop plant.

97. A transgenic plant according to claim 96, wherein said plant is tobacco or barley.

98. A method for reducing the amount of ROS in a plant in response to stress, comprising expressing a nucleic acid encoding a flavodoxin polypeptide and a nucleic acid encoding ferredoxin NADP(H) reductase polypeptide in a plant, wherein said nucleic acid sequences are of bacterial origin.

99. A method for the production of a product comprising the steps of growing a plant or plant part according to claim 94 and producing said product from or by the plants or parts.

100. A method for increasing the stress response or tolerance of a plant, comprising expressing a nucleic acid sequence encoding a flavodoxin polypeptide and a nucleic acid sequence encoding a ferredoxin NADP(H) reductase polypeptide in a plant, wherein said nucleic acid sequences are of bacterial origin.


SEQUENCE LISTING (12 sequences)

SEQ ID NO: 1 - DNA, 513 bp, Cyanobacteria
atgtcaaagaaaattggtttattctacggtactcaaactggtaaaactgaatcagtagcagaaatcattcgagacgagtttggtaatgatgtggtgacattacacgatgtttcccaggcagaagtaactgacttgaatgattatcaatatttgattattggctgtcctacttggaatattggcgaactgcaaagcgattgggaaggactctattcagaactggatgatgtagattttaatggtaaattggttgcctactttgggactggtgaccaaataggttacgcagataattttcaggatgcgatcggtattttggaagaaaaaatttctcaacgtggtggtaaaactgtcggctattggtcaactgatggatatgattttaatgattccaaggcactaagaaatggcaagtttgtaggactagctcttgatgaagataatcaatctgacttaacagacgatcgcatcaaaagttgggttgctcaattaaagtctgaatttggtttgtaa

SEQ ID NO: 2 - DNA, 675 bp, Artificial
  Fld nucleic acid sequence for use in single fusion construct with targeting sequence
atggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctctcataatgtcaaagaaaattggtttattctacggtactcaaactggtaaaactgaatcagtagcagaaatcattcgagacgagtttggtaatgatgtggtgacattacacgatgtttcccaggcagaagtaactgacttgaatgattatcaatatttgattattggctgtcctacttggaatattggcgaactgcaaagcgattgggaaggactctattcagaactggatgatgtagattttaatggtaaattggttgcctactttgggactggtgaccaaataggttacgcagataattttcaggatgcgatcggtattttggaagaaaaaatttctcaacgtggtggtaaaactgtcggctattggtcaactgatggatatgattttaatgattccaaggcactaagaaatggcaagtttgtaggactagctcttgatgaagataatcaatctgacttaacagacgatcgcatcaaaagttgggttgctcaattaaagtctgaatttggtttgtaa

SEQ ID NO: 3 - DNA, 915 bp, Anabaena PCC7119
atgactcaagcgaaagccaaacacgctgatgttcctgttaatctttaccgtcccaatgctccatttattggtaaggtaatctctaatgaaccactggtaaaagaaggcgggataggtattgttcagcacattaaatttgatctaactggtggtaacttaaagtacatcgaaggtcaaagtattggtatcattccaccaggagtggacaagaacggcaagccggaaaaattgagactctactccattgcctcgacccgtcacggcgatgatgtggatgataaaaccatctcactgtgcgtccgtcaattagagtacaaacatccagaaagcggcgaaacagtttacggtgtttgttctacttacttgactcacattgaaccaggttcagaagtgaaaatcactgggcctgtgggtaaagaaatgctgttacccgatgatcctgaagctaatgtcatcatgttggcaacaggtactggtattgcgcctatgcggacttacctgtggcggatgttcaaggatgcagaaagagctgctgacccagaatatcaattcaaaggattctcttggttagtctttggtgttcctacaactcctaacattctttataaagaagaactggaagaaatccaacaaaaatatcccgataacttccgcctaacttacgctatcagccgggagcaaaagaatccccaaggtggcagagtgtacatccaagaccgtgtggcagaacacgctgatgaactgtggcaattaatcaagaatgaaaaaacccacacctacatctgtggtttgcgcggtatggaagagggcattgatgctgctttaagtgctgcggctgcgaaagaaggtgttacctggagtgattaccaaaaagacctcaagaaagctggtcgctggcacgtagaaacatactaa

SEQ ID NO: 4 - DNA, 1077 bp, Artificial
  FNR nucleic acid sequence for use in single fusion construct (FNR construct with sequence encoding two domains) with targeting sequence.
atggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctcacaccatgactcaagcgaaagccaaacacgctgatgttcctgttaatctttaccgtcccaatgctccatttattggtaaggtaatctctaatgaaccactggtaaaagaaggcgggataggtattgttcagcacattaaatttgatctaactggtggtaacttaaagtacatcgaaggtcaaagtattggtatcattccaccaggagtggacaagaacggcaagccggaaaaattgagactctactccattgcctcgacccgtcacggcgatgatgtggatgataaaaccatctcactgtgcgtccgtcaattagagtacaaacatccagaaagcggcgaaacagtttacggtgtttgttctacttacttgactcacattgaaccaggttcagaagtgaaaatcactgggcctgtgggtaaagaaatgctgttacccgatgatcctgaagctaatgtcatcatgttggcaacaggtactggtattgcgcctatgcggacttacctgtggcggatgttcaaggatgcagaaagagctgctgacccagaatatcaattcaaaggattctcttggttagtctttggtgttcctacaactcctaacattctttataaagaagaactggaagaaatccaacaaaaatatcccgataacttccgcctaacttacgctatcagccgggagcaaaagaatccccaaggtggcagagtgtacatccaagaccgtgtggcagaacacgctgatgaactgtggcaattaatcaagaatgaaaaaacccacacctacatctgtggtttgcgcggtatggaagagggcattgatgctgctttaagtgctgcggctgcgaaagaaggtgttacctggagtgattaccaaaaagacctcaagaaagctggtcgctggcacgtagaaacatactaa

SEQ ID NO: 5 - DNA, 1323 bp, Cyanobacteria
atgtctaatcaaggtgcttttgatggtgctgccaacgtagaatcaggtagccgcgtcttcgtttacgaagtggtgggtatgcgtcagaacgaagaaactgatcaaacgaactacccaattcgtaaaagtggcagtgtgttcattagagtgccttacaaccgcatgaatcaagaaatgcagcgtatcactcgactaggcggcaagattgttacgattcaaacagtaagcgcactacaacaactcaatggtagaactaccattgcaacagtaacagatgcgtctagtgagattgctaagtctgaggggaatggtaaagccacacctgtaaaaactgatagtggagctaaagcgttcgctaaaccaccagctgaagaacagcttaagaaaaaagacaacaaaggcaacaccatgactcaagcgaaagccaaacacgctgatgttcctgttaatctttaccgtcccaatgctccatttattggtaaggtaatctctaatgaaccactggtaaaagaaggcgggataggtattgttcagcacattaaatttgatctaactggtggtaacttaaagtacatcgaaggtcaaagtattggtatcattccaccaggagtggacaagaacggcaagccggaaaaattgagactctactccattgcctcgacccgtcacggcgatgatgtggatgataaaaccatctcactgtgcgtccgtcaattagagtacaaacatccagaaagcggcgaaacagtttacggtgtttgttctacttacttgactcacattgaaccaggttcagaagtgaaaatcactgggcctgtgggtaaagaaatgctgttacccgatgatcctgaagctaatgtcatcatgttggcaacaggtactggtattgcgcctatgcggacttacctgtggcggatgttcaaggatgcagaaagagctgctgacccagaatatcaattcaaaggattctcttggttagtctttggtgttcctacaactcctaacattctttataaagaagaactggaagaaatccaacaaaaatatcccgataacttccgcctaacttacgctatcagccgggagcaaaagaatccccaaggtggcagagtgtacatccaagaccgtgtggcagaacacgctgatgaactgtggcaattaatcaagaatgaaaaaacccacacctacatctgtggtttgcgcggtatggaagagggcattgatgctgctttaagtgctgcggctgcgaaagaaggtgttacctggagtgattaccaaaaagacctcaagaaagctggtcgctggcacgtagaaacatactaa

SEQ ID NO: 6 - protein, 170 aa, Cyanobacteria
MSKKIGLFYGTQTGKTESVAEIIRDEFGNDVVTLHDVSQAEVTDLNDYQYLIIGCPTWNIGELQSDWEGLYSELDDVDFNGKLVAYFGTGDQIGYADNFQDAIGILEEKISQRGGKTVGYWSTDGYDFNDSKALRNGKFVGLALDEDNQSDLTDDRIKSWVAQLKSEFGL

SEQ ID NO: 7 - protein, 224 aa, Artificial
  Fld amino acid sequence with targeting sequence
MAAAVTAAVSLPYSNSTSLPIRTSIVAPERLVFKKVSLNNVSISGRVGTIRALIMSKKIGLFYGTQTGKTESVAEIIRDEFGNDVVTLHDVSQAEVTDLNDYQYLIIGCPTWNIGELQSDWEGLYSELDDVDFNGKLVAYFGTGDQIGYADNFQDAIGILEEKISQRGGKTVGYWSTDGYDFNDSKALRNGKFVGLALDEDNQSDLTDDRIKSWVAQLKSEFGL

SEQ ID NO: 8 - protein, 304 aa, Anabaena PCC7119
MTQAKAKHADVPVNLYRPNAPFIGKVISNEPLVKEGGIGIVQHIKFDLTGGNLKYIEGQSIGIIPPGVDKNGKPEKLRLYSIASTRHGDDVDDKTISLCVRQLEYKHPESGETVYGVCSTYLTHIEPGSEVKITGPVGKEMLLPDDPEANVIMLATGTGIAPMRTYLWRMFKDAERAADPEYQFKGFSWLVFGVPTTPNILYKEELEEIQQKYPDNFRLTYAISREQKNPQGGRVYIQDRVAEHADELWQLIKNEKTHTYICGLRGMEEGIDAALSAAAAKEGVTWSDYQKDLKKAGRWHVETY

SEQ ID NO: 9 - protein, 358 aa, Anabaena PCC7119
MAAAVTAAVSLPYSNSTSLPIRTSIVAPERLVFKKVSLNNVSISGRVGTIRAHTMTQAKAKHADVPVNLYRPNAPFIGKVISNEPLVKEGGIGIVQHIKFDLTGGNLKYIEGQSIGIIPPGVDKNGKPEKLRLYSIASTRHGDDVDDKTISLCVRQLEYKHPESGETVYGVCSTYLTHIEPGSEVKITGPVGKEMLLPDDPEANVIMLATGTGIAPMRTYLWRMFKDAERAADPEYQFKGFSWLVFGVPTTPNILYKEELEEIQQKYPDNFRLTYAISREQKNPQGGRVYIQDRVAEHADELWQLIKNEKTHTYICGLRGMEEGIDAALSAAAAKEGVTWSDYQKDLKKAGRWHVETY

SEQ ID NO: 10 - protein, 440 aa, Cyanobacteria
MSNQGAFDGAANVESGSRVFVYEVVGMRQNEETDQTNYPIRKSGSVFIRVPYNRMNQEMQRITRLGGKIVTIQTVSALQQLNGRTTIATVTDASSEIAKSEGNGKATPVKTDSGAKAFAKPPAEEQLKKKDNKGNTMTQAKAKHADVPVNLYRPNAPFIGKVISNEPLVKEGGIGIVQHIKFDLTGGNLKYIEGQSIGIIPPGVDKNGKPEKLRLYSIASTRHGDDVDDKTISLCVRQLEYKHPESGETVYGVCSTYLTHIEPGSEVKITGPVGKEMLLPDDPEANVIMLATGTGIAPMRTYLWRMFKDAERAADPEYQFKGFSWLVFGVPTTPNILYKEELEEIQQKYPDNFRLTYAISREQKNPQGGRVYIQDRVAEHADELWQLIKNEKTHTYICGLRGMEEGIDAALSAAAAKEGVTWSDYQKDLKKAGRWHVETY

SEQ ID NO: 11 - DNA, 27 bp, Artificial
  Primers for amplification of FNR region
ccgagctcacaccatgactcaagcgaa

SEQ ID NO: 12 - DNA, 27 bp, Artificial
  Primers for amplification of FNR region
acgtcgaccaacttagtatgtttctac